(19) United States
(12) Patent Application Publication
(10) Pub. No.: US 2024/0344053 A1
(43) Pub. Date: Oct. 17, 2024

(54) Self-immolative plasmid backbone

(71) Applicant: ProteoNic Biotechnology IP B.V., Leiden (NL)

(72) Inventors: Bart Marinus Engels, Woerden (NL); Raymond Michael Dimphena Verhaert, Breda (NL); Maurice Wilhelmus Van der Heijden, Waddinxveen (NL)

(73) Assignee: ProteoNic Biotechnology IP B.V., Leiden (NL)

(21) Appl. No.: 18/602,065

(22) Filed: Mar. 12, 2024

Related U.S. Application Data
(62) Division of application No. 17/433,609, filed on Aug. 25, 2021, now Pat. No. 11,959,069.

(30) Foreign Application Priority Data
Feb. 28, 2019 (EP) 19160099.8

(51) Int. Cl. C12N 15/10

(52) U.S. Cl. CPC C12N 15/101

(57) ABSTRACT

The invention relates to a method for separating a polynucleotide insert from a polynucleotide vector backbone. The backbone has a plurality of cleavage sites distributed such that the backbone is converted into fragments when the sites are cleaved. This allows straightforward separation of the insert from the backbone. The invention also relates to backbones for use in such a method, and to plasmids and kits comprising such backbones.

### REFERENCE TO AN ELECTRONIC SEQUENCE LISTING

The contents of the electronic sequence listing (sequence Listing—st26.xml; Size: 449 Kb; and Date of Creation: May 28, 2024) is herein incorporated by reference in its entirety

### FIELD OF THE INVENTION

The invention is in the field of polynucleotides and their production. The invention relates to a method for separating a polynucleotide insert from a polynucleotide vector backbone. The backbone has a plurality of cleavage sites distributed such that the backbone is converted into fragments when the sites are cleaved. This allows straightforward separation of the insert from the backbone. The invention also relates to backbones for use in such a method, and to plasmids and kits comprising such backbones.

### BACKGROUND ART

Some uses of polynucleotides such as DNA require that a DNA fragment of interest is preserved and purified while other fragments are not needed and therefore are to be discarded. This can be the case when high purity polynucleotides are required, or when high efficiency of polynucleotide handling is required where contaminations would reduce handling efficiency. Separation of polynucleotides with a desired sequence from undesired polynucleotides is an essential, common process that is routinely carried out in molecular biology. Typically a fragment of interest (FOI) is present in a vector which is propagated in a bacterium. When this FOI is needed for later use, it needs to be separated from the remainder of the vector, which is generally a bacterial sequence. This time consuming effort requires molecular cloning activities, and is required to optimize the results of any follow-up experiment carried out with the purified polynucleotide.

In conventional purification of an insert from a vector, typically, restriction enzymes are used that allow the polynucleotide to be digested resulting in two fragments: a desired fragment and an unwanted fragment. After the actual digestion reaction has been carried out, the reaction mixture is loaded on an agarose gel to separate the fragments based on their size-determined difference in migration, and the desired fragment is subsequently collected from the agarose gel by cutting out the proper gel piece. The insert is then purified from the agarose gel material by centrifugation and precipitation, often using specific gel-dissolving solutions. While it is a routine process, this protocol is cumbersome and time consuming, which can lead to lower yields of desired fragments than theoretically possible. Additionally, preparation of desired polynucleotide often is further complicated when the size difference between the desired fragment and the undesired fragment is small, for in that case a more specific digestion protocol needs to be developed and carried out. Thus there is a need for a system that limits the amount of steps.

Besides common cloning activities, large scale polynucleotide isolations also often require removal of undesired polynucleotide fragments. For instance, for gene therapy applications significant amounts of DNA are desired from which part of the plasmid is removed out of safety concerns (antibiotic resistance markers [New Generation of Plasmid Backbones Devoid of Antibiotic Resistance Marker for Gene Therapy Trials. G Vandermeulen et al., Mol Therapy 2011 19, 1942-1949.], induced inflammatory responses [An araC-controlled bacterial cre expression system to produce DNA minicircle vectors for nuclear and mitochondrial gene therapy. BW Bigger et al., J Biol Chem. (2001) 276, 23018-23027.]), or because it negatively affects gene performance. Thus extensive research has been devoted to develop DNA material in which parts of a plasmid are removed. For example the resistance marker can be removed (see above Vandermeulen et al,) or both the resistance marker(s) as well as the genetic material that is essential to produce the plasmid in a microorganism can be removed. DNA fragments devoid of bacterial selection markers and/or all bacterial backbone sequences also are known to improve the expression of the eukaryotic gene that is to be expressed in a cultured mammalian host cell.

Currently, specific bacterial cell hosts are used to generate miniplasmid DNA. Following the production of a plasmid it is opened inside the cell with a specific enzyme. After removal of undesired sequences it is recicularised to form a miniplasmid. The DNA that is obtained after opening the bacterial cells needs to be purified from original full size plasmids and DNA debris. Combining efficient in vitro isolation of a desired DNA fragment with degradation and elimination of the backbone offers a simplification and an improvement of the process.

An improved method for separating polynucleotide fragments of interest from unwanted sections of polynucleotide is highly desired, for instance to facilitate isolation of an insert from a vector backbone. Polynucleotides for use in such improved methods are also desired.

### SUMMARY OF THE INVENTION

The invention relates to a backbone for use in a vector, primed to allow the backbone to be degraded into small fragments by using cleavage means (e.g. restriction enzymes), while leaving the insert of the vector intact. The cleavage sites in the backbone are separated in such a way that the backbone is divided into small fragments. Such cleavage sites are not present in the insert. As a result, treatment of the vector backbone with for example the appropriate restriction enzymes will leave the insert as the sole polynucleotide fragment of considerable length. This enables easy separation between the desired fragment and the undesired backbone debris.

Accordingly, in a first aspect the invention provides a method for separating a polynucleotide insert from a polynucleotide vector backbone, the method comprising the steps of
i) providing a recombinant polynucleotide vector comprising the insert and the vector backbone, wherein the vector backbone comprises a first plurality of cleavage sites that divide the vector backbone into fragments having a length of at most 1000 bp;
ii) contacting the recombinant vector with cleavage means capable of specifically cleaving the first plurality of cleavage sites to produce backbone fragments; and optionally,
iii) separating the insert from the backbone fragments of step ii).
   In preferred embodiments the separation of step iii uses a technique selected from a spin column, a size exclusion column, and solid phase reversible immobilization (SPRI). In preferred embodiments the cleavage means are selected from the group consisting of a restriction enzyme, an RNA-guided DNA endonuclease enzyme, a sequence-specific nuclease, sequence-specific ultrasonication, a sequence-specific oxidative small molecule such as bleomycin, and a sequence-specific hydrolyzing small molecule such as a lanthanide complex. In more preferred embodiments the cleavage sites are restriction endonuclease recognition sites. Preferably, the cleavage means comprise 3, 2, or 1 species of restriction enzymes, preferably wherein the restriction enzyme recognizes a restriction site of 6 or 7 nucleotides, more preferably selected from the group consisting of BstZ17I and MluI. Preferably, the insert does not comprise a cleavage site of the first plurality of cleavage sites. Preferably, the vector backbone further comprises a polynucleotide encoding a functional selection marker, wherein the polynucleotide encoding a functional selection marker is preferably selected from SEQ ID NOs: 14-18, 79, and 110-116, or from a polynucleotide encoding a selection marker selected from SEQ ID NOs: 69, 70, and 81-88. Preferably, the fragments have a length of at most 900, 800, 700, 650, 600, 550, 500, 450, 400, 350, 300, 250, 200, 150, or 100 bp or less, preferably of at most 550 bp, such as about 500 bp. In preferred embodiments is provided the method wherein the vector backbone comprises a multiple cloning site that does not comprise a cleavage site of the first plurality of cleavage sites.

In a second aspect is provided a polynucleotide vector backbone as defined in the first aspect. Preferably, the vector backbone has at least 70% sequence identity with any one of SEQ ID NOs: 68 and 80. In preferred embodiments is provided a recombinant polynucleotide vector comprising a polynucleotide insert and a polynucleotide vector backbone as defined above.

In a third aspect is provided a method for amplifying a polynucleotide of interest, the method comprising the steps of: i) providing a recombinant polynucleotide vector comprising the polynucleotide of interest as an insert, and a vector backbone according to the second aspect; ii) amplifying the recombinant polynucleotide vector of step i) by transforming a suitable microorganism with it, and culturing said transformed microorganism in a culture medium under conditions suitable for vector amplification to obtain amplified recombinant polynucleotide vector; iii) isolating the amplified recombinant polynucleotide vector from the culture medium of step ii) to obtain isolated amplified recombinant polynucleotide vector; iv) contacting the isolated amplified recombinant polynucleotide vector with cleavage means capable of cleaving the first plurality of cleavage sites to produce backbone fragments; and optionally, v) separating the nucleotide sequence of interest from the backbone fragments of step iv).

In a fourth aspect the invention provides a kit of parts comprising: i) a polynucleotide vector backbone according to the second aspect, and at least one of iia) materials for use in a separation technique as defined in the first aspect, such as spin filters; or iib) cleavage means as defined in the first aspect.

In a fifth aspect the invention provides the use of a polynucleotide vector backbone according to the second aspect for the purification of a polynucleotide insert. In preferred embodiments this use is in a method for enhancing transcription of a nucleotide sequence of interest in a eukaryotic cell, the method comprising the steps of: i) providing a recombinant polynucleotide vector comprising the polynucleotide of interest as an insert, and a vector backbone according to the second aspect; ii) amplifying the recombinant polynucleotide vector of step i) by transfecting it to a suitable microorganism and culturing said microorganism in a culture medium under conditions suitable for vector amplification to obtain amplified recombinant polynucleotide vector; iii)isolating the amplified recombinant polynucleotide vector from the culture medium of step ii) to obtain isolated amplified recombinant polynucleotide vector; iv) contacting the isolated amplified recombinant polynucleotide vector with cleavage means capable of cleaving the first plurality of cleavage sites to produce backbone fragments; v) separating the nucleotide sequence of interest from the backbone fragments of step iv) to obtain an isolated nucleotide sequence of interest; vi) integrating the isolated nucleotide sequence of interest in the genome of a eukaryotic cell, preferably a mammalian cell or an insect cell, to obtain a transgenic cell; vii) culturing the transgenic cell under conditions conducive to expression of the nucleotide sequence of interest.

### DESCRIPTION OF EMBODIMENTS

### Self-Immolative Polynucleotide Vector Backbone

The invention relates to a backbone that can be used in a vector, which is primed to allow the backbone to be shredded by the use of cleavage means such as restriction enzymes, while leaving the insert (which generally comprises a region of interest) intact. The backbone is divided into small fragments by cleavage sites, which are not present in the insert, and thus the vector backbone is primed for degradation. After cleavage the insert remains as the sole polynucleotide fragment of considerable length, enabling easy separation between the desired fragment and the undesired debris that remains of the vector backbone. The invention is easily implemented without the requirement of additional hardware, and can be practiced in any lab equipped for molecular cloning. The self-immolative backbone can be used in methods for the production of polynucleotide fragments. Such methods can also be used for efficient preparation of specific homology-directed repair (HDR) template DNA in CRISPR/Cas mediated DNA-editing, or for preparation of RNA for use in synthetic biology. Such methods can also be used to prepare (circular) RNA for use in e.g. synthetic biology. The method is easier and faster than existing methods and leads to improved insert yields.

In a first aspect the invention provides a polynucleotide vector backbone, wherein the vector backbone comprises a first plurality of cleavage sites that divide the vector backbone into fragments having a length of at most 1000 bp. Such a polynucleotide vector backbone is referred to herein as a backbone according to the invention. An insert that is intended to be used with the backbone is similarly referred to as an insert for use in the invention; a vector comprising a backbone according to the invention is referred to as a vector according to the invention. Because the backbone can disassemble into fragments, it is reminiscent of synthetic self-immolative polymers that disassemble into monomers when a trigger is provided. While the backbone does not disassemble into monomers, it does disassemble into fragments that are sufficiently small to be easily disposed of. For ease of reference, sequences of polynucleotide that together constitute a single fragment (that is: a discrete molecule) after degradation are also referred to as a fragment when the intact backbone is discussed (that is: when the fragments are still joined in the backbone). It will be apparent from context whether a fragment refers to a discrete polynucleotide molecule that is a degradation product of a backbone according to the invention, or whether it refers to a polynucleotide sequence in between two cleavage sites in the intact backbone. FIG. 1 illustrates this aspect.

A vector, sometimes referred to as a plasmid, is herein understood to mean a man-made, or recombinant, nucleic acid molecule resulting from the use of recombinant polynucleotide technology, and which for example can be used to deliver exogenous DNA into a host cell. As used herein, “vector” and “recombinant vector” are used interchangeably as non-recombinant vectors are not part of the invention. Vectors are preferably circular, and usually comprise further genetic elements to facilitate their use in molecular cloning, such as for example selectable markers, multiple cloning sites, et cetera. A vector is said to be recombinant when it comprises sequences that originally derive from different sources, such as from different organisms. The vector is a polynucleotide, which means that it can be DNA or RNA. Because it is more convenient to use in practice, in preferred embodiments the polynucleotide is DNA.

A vector is generally used to either amplify a specific sequence of interest, or to isolate, identify, and express a sequence of interest, or to express one. The vector is then designed around this sequence of interest, with said sequence being referred to as an insert, and with the remainder of the vector being referred to as the backbone of the vector. Often the insert in a plasmid represents exogenous DNA while the backbone of the plasmid facilitates its use and multiplication. This is known to a skilled person. In general, a vector consists of a backbone (which is responsible for functionality in that it harbours features such as a selection marker or a multiple cloning site or an origin of replication) and an insert, which is the polynucleotide that comprises the sequence of interest. It can for example encode a gene of interest. However, some vectors do not comprise an insert. For example, for production of the backbone it is convenient to amplify a plasmid consisting only of the backbone. This is often referred to as an empty vector. Through standard cloning techniques an insert can be incorporated, for example in the multiple cloning site. Because of this close interrelation, any reference to a vector can also be read as a reference to the backbone of that vector, and conversely any reference to a backbone can be read as a reference to a vector, particularly to an empty vector, such as shown in FIG. 2.

Many vectors and their accompanying backbones are known in the art, and are commercially available. A vector may for example be part of a recombinant viral vector for expression of a protein in a plant or plant cell (e.g. a vector derived from cauliflower mosaic virus, CaMV, or tobacco mosaic virus, TMV) or in a mammalian organism or mammalian cell system (e.g. a vector derived from Moloney murine leukemia virus (MMLV), a Retrovirus, a Lentivirus, an Adeno-associated virus (AAV) or an adenovirus (AdV)). Accordingly a preferred backbone is a viral backbone, such as a CaMV, TMV, MMLV, retroviral, lentiviral, AAV, or AdV backbone. In preferred embodiments of this aspect, the backbone according to the invention is comprised in a recombinant polynucleotide vector; accordingly, in preferred embodiments the invention provides a recombinant polynucleotide vector comprising a polynucleotide insert and a backbone according to the invention. More preferred embodiments consist of such a backbone and such an insert. It is to be understood that when a vector is said to consist of a given set of elements, this does not imply that some short linker sequences or individual base pairs, for example vestiges from a cloning process, cannot be present at all; rather it is to be understood that a vector consisting of specific elements does not comprise any substantial further elements.

Examples of suitable backbones according to the invention are shown in the examples and provided in SEQ ID NOs: 68 and 80. Examples of suitable backbones that can be modified by introduction of cleavage sites, thus forming backbones according to the invention, are provided in SEQ ID NOs: 1-6. A skilled person knows that silent mutations can be made in such backbones, leading to identical or substantially identical functionality; and that rearrangement of functional units within the backbone can be made, or that individual units can be inverted, or that one functional unit such as a marker can be replaced by a different functional unit of the same type such as a different marker, or combinations of any of these changes. Therefore, in preferred embodiments is provided a polynucleotide vector backbone according to the invention, wherein the vector backbone has at least 70%, 75%, 80%, 85%, 90%, 91%, 92%, 93%, 94%, 95%, 96%, 97%, 98%, 99%, or 100% sequence identity with any one of SEQ ID NOs: 1-6, 68, and 80, preferably 68 and 80; preferably having at least 90%, 91%, 92%, 93%, 94%, 95%, 96%, 97%, 98%, 99%, or 100% sequence identity with any one of SEQ ID NOs: 1-6, 68, and 80, preferably 68 and 80; more preferably having at least 95%, 96%, 97%, 98%, 99%, or 100% sequence identity with any one of SEQ ID NOs: 1-6, 68, and 80, preferably 68 and 80; most preferably having at least 98%, 99%, or 100% sequence identity with any one of SEQ ID NOs: 1-6, 68, and 80, preferably 68 and 80.

To prevent its degradation, it is preferred that the insert in such a vector not have a cleavage site that is involved in the immolation of the backbone, accordingly, in preferred embodiments is provided a recombinant polynucleotide vector comprising a polynucleotide insert and a backbone according to the invention, wherein the insert does not comprise a cleavage site of the first plurality of cleavage sites; more preferably the insert does not comprise a species of cleavage site of the first plurality of cleavage sites.

### Cleavage Sites, Cleavage Means, and Fragments

To enable its use, the backbone according to the invention has a first plurality of cleavage sites. A cleavage site is a well-defined region of the backbone that can be cleaved by controlled means. A very common example of a cleavage site is a restriction site. Many such cleavage sites are known in the art. A cleavage site is often a particular short sequence, and can be cleaved, for example hydrolyzed, by a cleavage means. For example, a restriction site of six nucleotides in length can be recognized for example by its recognition sequence, and then cleaved by a restriction enzyme. As is known in the art, this cleavage can be inside the recognition site, or at one of its extremes, or even one or two or more nucleotides away from the site. As is known, a cleavage site can in some cases be non-contiguous, as for example is the case for restriction enzyme AccB7I, which recognizes CCA directly followed by any five nucleotides, directly followed by TGG; such a cleavage site has a restriction recognition site of six defined nucleotides, yet it is a cleavage site of 11 nucleotides in length. The cleavage means recognizes the cleavage site (most preferably because of a recognition sequence) and for example causes a double-stranded chain break, cutting the polynucleotide at the cleavage site. Of course, a cleavage site can be such that the actual chain break is at an area outside the actual recognition sequence, but it is common to still refer to cleavage induced by such a site as “cleavage of that site”. Each cleavage site has its associated cleavage means. A cleavage site preferably has a length of at least 3, 4, 5, or 6 nucleotides, more preferably at least 4, most preferably at least 5 nucleotides. A cleavage site preferably has a recognition sequence that has a length of at least 3, 4, 5, or 6 nucleotides, more preferably at least 4, even more preferably at least 5, most preferably at least 6 nucleotides. A cleavage site or a recognition sequence preferably has a length of at most 20 nucleotides, more preferably at most 15, even more preferably at most 10, still more preferably at most 9, still more preferably at most 8 nucleotides.

Types of cleavage site, such as restriction sites, can have multiple species, such as restriction sites that are recognized by different restriction enzymes. Another example is that the type of cleavage site that is RNA-guided DNA endonuclease recognition sites, has multiple species of cleavage site, such as cleavage sites that are recognized by RNA-guided DNA endonuclease enzymes comprising different guide RNAs.

In preferred embodiments, the cleavage means are selected from the group consisting of restriction enzymes, RNA-guided DNA endonuclease enzymes, sequence-specific nucleases, sequence-specific ultrasonication, sequence-specific oxidative small molecules such as bleomycin, and sequence-specific hydrolyzing small molecules such as lanthanide complexes. More preferred cleavage means are selected from the group consisting of restriction enzymes, RNA-guided DNA endonuclease enzymes, and sequence-specific nucleases. Restriction enzymes are most preferred. Accordingly, in preferred embodiments is provided a backbone according to the invention, wherein the cleavage sites from the first plurality of cleavage sites are restriction endonuclease recognition sites.

Restriction enzymes are broadly known in the art and are widely commercially available (see e.g. Loenen et al., Nucleic Acids Res. 2014 42(1): 3-19 DOI: 10.1093/nar/gkt990). They are also known as restriction endonucleases. A restriction enzyme or restriction endonuclease is an enzyme that cleaves polynucleotides such as DNA into fragments at or near a specific recognition site. A restriction site, or restriction recognition site, or a restriction endonuclease recognition site, is a location on a DNA molecule containing a specific sequence of nucleotides, typically 4-8 base pairs in length and often palindromic, that is recognized by a restriction enzyme. Preferred restriction sites are non-contiguous restriction sites because they allow non-defined nucleotide positions; in preferred embodiments, the first plurality of cleavage sites comprises at least one non-contiguous restriction site. Examples of restriction enzymes are AanI, AarI, AasI, AatI, AatII, AbsI, AccI, AccIII, Acc16I, Acc36I, Acc65I, Acc113I, AccB1I, AccB7I, AccBSI, AciI, AcII, AcIWI, AcoI, AcsI, AcuI, AcvI, AcyI, AdeI, AfaI, AfeI, AfiI, AflIII, AgeI, AhdI, AhII, AjiI, AjnI, AjuI, AleI, AlfI, AloI, AluI, AluBI, AlwI, Alw21I, Alw26I, Alw44I, AlwNI, Ama87I, Aor13HI, Aor51HI, ApaI, ApaLI, ApeKI, ApoI, AscI, AseI, AsiGI, AsiSI, Asp700I, Asp718I, AspA2I, AspEI, AspLEI, AspS9I, AsuII, AsuC2I, AsuHPI, AsuNHI, AvaI, AvaII, AviII, AvrII, AxyI, BaeI, BaeGI, BaII, BamHI, BanI, BanII, BanlII, BarI, BauI, BbeI, BbrPI, BbsI, BbuI, BbvI, Bbv12I, BbvCI, BccI, BceAI, BcgI, BciVI, BciT130I, BcII, BcnI, BcoDI, BcuI, BdaI, BfaI, BfiI, BfmI, BfoI, BfuI, BfuAI, BfuCI, BgII, BglII, BisI, BlnI, BIpI, BIsI, BmcAI, Bme18I, Bme1390I, Bme1580I, BmeRI, BmeT110I, BmgBI, BmgT120I, BmiI, BmrI, BmrFI, BmsI, BmtI, BmuI, BoxI, BpiI, Bpli, BpmI, Bpu10I, Bpu14I, Bpu1102I, BpuAi, BpuEI, BpuMI, BpvUI, BsaI, Bsa29I, BsaAI, BsaBI, BsaHi, BsaJI, BsaMI, BsaWi, BsaXI, Bsc4I, Bse1I, Bse3DI, Bse8I, Bse21I, Bse118I, BseAI, BseBI, BseCi, BseDI, BseGI, BseJi, BseLi, BseMI, BseMII, BseNI, BsePI, BseRI, BseSI, BseXI, BseX3I, BseYI, BsgI, BshVI, Bsh1285I, BshFI, BshNI, BshTI, BsiEI, BsiHKAI, BsiHKCI, BsiSI, BsiWI, BsiYI, BsII, BsIFI, BsmI, BsmAI, BsmBI, BsmFI, BsnI, Bso31I, BsoBI, Bsp13I, Bsp19I, Bsp68I, Bsp119I, Bsp120I, Bsp143I, Bsp1286I, Bsp1407I, Bsp1720I, BspACI, BspCNI, BspDI, BspEI, BspHI, BspLI, BspLU11I, BspMI, BspOi, BspPI, BspQI, BspTI, BspT104I, BspT107I, BspTNI, BspXI, BsrI, BsrBI, BsrDI, BsrFI, BsrGI, BsrSI, BssAI, BssECI, BssHII, BssKI, BssMI, BssNI, BssNAI, BssSaI, BssT1I, Bst2BI, Bst2UI, Bst4CI, Bst6I, Bst1107I, BstACI, BstAPI, BstAUI, BstBI, BstBAI, BstC8I, BstDEI, BstDSI, BstEII, BstENI, BstF5I, BstH2I, BstHHI, BstHPI, BstKTI, BstMAI, BstMBI, BstMCI, BstMWI, BstNI, BstNSI, BstOI, BstPI, BstPAI, BstSCI, BstSFI, BstSLI, BstSNI, BstV1I, BstV2I, BstXI, BstX2I, BstYI, BstZI, BstZ17I, Bsu15I, Bsu36I, BsuRI, BsuTUI, BtgI, BtgZI, BtrI, BtsCI, BtslMutI, BtsaI, BtuMI, BveI, Cac8I, CaiI, CciI, CciNI, CeIi, CfoI, CfrI, Cfr9I, Cfr10I, Cfr13I, Cfr42I, ClaI, CpoI, CsiI, CspI, Csp6I, Csp45I, CspAI, CspCI, CviJI, CviKI-1, CviQI, DdeI, DinI, DpnI, DpnII, DraI, DraII, DraIII, DrdI, DriI, DseDI, EaeI, EagI, Eam1104I, Eam1105I, EarI, EciI, Ecl1361I, EclXI, Eco24I, Eco31I, Eco32I, Eco47I, Eco4711I, Eco52I, Eco53kI, Eco57I, Eco57MI, Eco72I, Eco81I, Eco88I, Eco91I, Eco105I, Eco130I, Eco147I, EcoICRI, EcoNI, EcoO65I, EcoO109I, EcoP15I, EcoRI, EcoRII, EcoRV, EcoT14I, EcoT22I, EcoT38I, EgeI, EheI, Erhi, Esp3I, FaII, FaqI, FauI, FauNDI, FbaI, Fbli, Fnu4HI, FokI, FriOI, FseI, Fsp4HI, FspI, FspAI, FspBI, GlaI, GiuI, GsaI, GsuI, HaeII, HaelII, HapII, HhaI, Hin1I, Hin4I, Hin6I, HinciI, HindII, HindIII, HinfI, HinP1I, HpaI, HpaII, HphI, Hpy8I, Hpy99I, Hpy1661I, Hpy188I, Hpy18811I, HpyAV, HpyCH411I, HpyCH4IV, HpyCH4V, HpyF3I, HpyF10VI, Hsp92I, HspAI, I-CeuI, I-PpoI, I-SceI, ItaI, KasI, KflI, KpnI, Kpn2I, KspI, Ksp22I, Ksp632I, KspAI, Kzo9I, LguI, Lsp1109I, LweI, MabI, MaeI, MaeII, MaII, MamI, MauBI, MbiI, MboI, MboII, MfeI, MfII, MhII, MisI, MiuI, MluCI, MluNI, MlyI, Mly113I, MmeI, MnII, Mph1103I, MreI, MroI, MroNI, MroXI, MscI, MsII, MspI, Msp20I, MspA1I, MspR9I, MssI, Muni, MvaI, Mva1269I, MwoI, NaeI, NarI, NciI, NcoI, NdeI, NdeII, NgoMIV, NheI, NlaIV, NmeAIII, NotI, NruI, NsbI, NsiI, NspI, NspV, OliI, PacI, PaeI, PaeR7I, PagI, PalAI, Pasi, PauI, PceI, PciI, PciSI, PctI, PdiI, PdmI, PfeI, Pfl231I, PfIMI, PfoI, PhoI, PI-PspI, PI-SceI, PinAI, PleI, Ple19I, PluTI, PmaCI, PmeI, PmII, PpiI, PpsI, Ppu21I, PpuMI, PscI, PshAI, PshBI, PsiI, Psp51I, Psp6I, Psp124BI, Psp1406I, PspCI, PspEI, PspFI, PspGI, PspLI, PspN4I, PspOMI, PspPI, PspPPI, PspXI, PsrI, PstI, PsuI, PsyI, PteI, PvuI, PvuII, RcaI, RgaI, Rigi, RruI, RsaI, RsaNI, RseI, RsrII, Rsr2I, SacI, SacII, SaII, SanDI, Sapi, Sati, Sau3AI, Sau96I, SbfI, ScaI, SchI, ScrFI, SdaI, SduI, Seti, SexAI, SfaAI, SfaNI, SfcI, SfiI, SfoI, Sfr274I, Sfr303I, SfuI, SgfI, SgrAI, SgrBI, SgrDI, SgsI, Sini, SlaI, SmaI, SmiI, SmiMI, SmoI, SmuI, SnaBI, SpeI, SphI, SrfI, Sse9I, Sse8387I, SseBI, SsiI, SspI, SspBI, SspDI, SstI, SstII, StuI, StyI, StyD4I, SwaI, TaaI, TaiI, TaqI, TaqII, TaqaI, TasI, Tati, TauI, TfiI, TiiI, TscAI, TseI, TsoI, Tsp509I, TspDTI, TspEI, TspGWI, TspMI, TspRI, TstI, Van91I, VneI, VpaK11BI, VspI, XagI, XapI, XbaI, XceI, XcmI, XhoI, XhoII, XmaI, XmaCI, XmaJI, XmiI, XmnI, XspI, ZraI, ZrmI, Zsp2I, and their isoschizomers or neoschizomers. Preferred restriction enzymes are endonucleases that recognise a DNA sequence, more preferably a DNA sequence of at least 6 nucleotides, even more preferably at least 6 or 7 nucleotides. Preferred restriction enzymes recognize a site of at most 7 nucleotides, more preferably of at most 6 nucleotides. Preferred restriction enzymes are not sensitive to methylation. Preferred restriction enzymes do not show star activity, more preferably do not show star activity under conditions used for the method according to the invention, even more preferably do not show star activity when no organic solvent is present, the ionic strength of the reaction buffer is at least 100 mM, and the pH of the reaction buffer is at most 7.6, most preferably do not show star activity at all. Restriction enzymes that do not see frequent use in routine cloning are very convenient because they would not interfere with common cloning processes involving a backbone according to the invention. Accordingly highly preferred restriction enzymes recognize at least 6 or 7, preferably 6 nucleotides and are not AgeI, BamHI, BssHII, DraI, EagI, EcoRI, EcoRV, HindIII, NcoI, PstI, PvuI, SalI, XbaI, XhoI, or XmaI, more preferably not AgeI, BamHI, BbsI, BfuAI, BglII, BsaI, BsmBI, BssHII, DraI, EagI, EcoRI, EcoRV, HindIII, NcoI, PstI, PvuI, SalI, XbaI, XhoI, or XmaI. Most preferred restriction enzymes for degrading the backbone according to the invention are BstZ17I and MluI.

A cleavage means can also be an RNA-guided DNA endonuclease enzyme, as used in the CRISPR/Cas technology and referred to hereinafter as a Cas endonuclease. A preferred Cas endonuclease is a Cas9 endonuclease. This technology is generally known, see for example Khan et al., Journal of Biomedical Science, 2018, 25:29, DOI: 10.1186/s12929-018-0425-5. A Cas endonuclease is guided by an RNA having any sequence of choice, and will cleave any cleavage site that its guide RNA points it to. This makes Cas endonucleases very versatile for cleaving a backbone according to the invention. Other Cas and Cas-like proteins can be used as well as different protospace adjacent motifs (PAMs).

Any other sequence-specific nuclease can be used to cleave its associated sequence. Zinc finger nucleases and TALEN belong to this group. An advantage of using restriction enzymes is their high fidelity of sequence specificity. Restriction enzymes have known, predictable and reliable specificity and fidelity.

Other suitable cleavage means are sequence-specific ultrasonication (see Grokhovsky et al., Biophys J. 2011; 100(1): 117-125; DOI: 10.1016/j.bpj.2010.10.052), a sequence-specific oxidative small molecule such as bleomycin or a modified porphyrin (see for example Van Dongen et al., Nature Chem. 5, p. 945-951 (2013) DOI: 10.1038/nchem.1752), and a sequence-specific hydrolyzing small molecule such as a lanthanide complex (see for example Hall et al., Nucleic Acids Res. 1996; 24(18): 3522-3526. PMID: 8836177). More preferred cleavage means are selected from the group consisting of a restriction enzyme, an RNA-guided DNA endonuclease enzyme, and a sequence-specific nuclease. A restriction enzyme is most preferred, for example because of its ready availability and convenient usability.

As mentioned above, a cleavage site is cleaved by its associated cleavage means. In the context of this document, cleavage is meant to refer to a break in the polynucleotide, which generally means a double-stranded break, as vectors and backbones are preferably double-stranded. In the context of this invention, cleavage refers to the controlled cleavage of a cleavage site, as effected by the associated cleavage means. The cleavage can be a direct double cleavage, leading to blunt ends, or it can be a set of two nicks in opposing strands, the nicks not being separated by more than about 15, preferably 10 nucleotides, leading to sticky ends. As long as adjoining fragments are no longer substantially associated with one another, the cleavage can be considered suitable for use in this invention. In preferred embodiments, cleavage is promoted by an increase in medium temperature or ionic strength, preferably medium temperature. Optionally, in these embodiments, additional agents may be added to increase dissociation of the cleaved fragments. Such agents are known in the art; examples are DMSO and formamide.

The first plurality of cleavage sites is configured to convert the backbone according to the invention to fragments. It is convenient when the cleavage sites in the first plurality can be cleaved in one single reaction step, possibly involving multiple different types or species cleavage means if the plurality comprises different types or species of cleavage sites. For example when the cleavage sites are restriction sites, the degradation can be performed by a mixture of restriction enzymes that can cleave their respective cleavage sites under the same reaction conditions. A mixture of cleavage means is not limited as to how many different cleavage means are comprised, as the invention relates to the degradation of the backbone and not in the details of how this is achieved precisely.

Nonetheless, for convenience it is preferred when the cleavage sites in the first plurality are not very diverse, and are generally of the same type. In preferred embodiments, the cleavage sites in the first plurality can all be cleaved under the same reaction conditions. For this reason, in preferred embodiments, all cleavage sites in the first plurality are restriction sites. In other preferred embodiments, all cleavage sites in the first plurality are recognition sites for an RNA-guided DNA endonuclease enzyme. In other preferred embodiments, all cleavage sites in the first plurality are recognition sites for a sequence-specific nuclease.

When the cleavage sites in the first plurality are all of the same type, it is preferred that the associated cleavage means is similarly of low diversity, and therefore substantially of the same species. For example, for ease of preparation, a mixture of cleavage means preferably comprises at most 5, 4, 3, 2, or 1 species of cleavage means, such as different restriction enzymes or RNA-guided DNA endonucleases with different guide RNAs. Accordingly, in preferred embodiments is provided the backbone according to the invention, wherein the cleavage sites comprise at most 5, 4, 3, 2, or 1 different cleavage sites, preferably at most 3, 2, or 1 different cleavage sites, more preferably at most 2 or 1 different cleavage sites, most preferably only 1 species of cleavage site.

In preferred embodiments, the first plurality of cleavage sites comprises only restriction sites, and comprises no more than 6, 5, 4, 3, 2, or 1, preferably no more than 3, 2, or 1, more preferably no more than 2 or 1, most preferably comprises only 1, species of restriction site, wherein the restriction sites are preferably selected from the group consisting of restriction sites for restriction enzymes as described above, more preferably selected from the group consisting of restriction sites for BssHI, BstZ17I, and MluI, most preferably selected from restriction sites for BstZ17I and MluI.

In preferred embodiments, the first plurality of cleavage sites comprises only recognition sites for an RNA-guided DNA endonuclease enzyme, and comprises no more than 5, 4, 3, 2, or 1, preferably no more than 3, 2, or 1, more preferably no more than 2 or 1, most preferably comprises only 1, species of recognition site.

### Degradation of the Backbone

As explained earlier herein, the invention revolves around a backbone that can be degraded into smaller fragments, to allow easy isolation of an insert. The vector backbone is divided into fragments by the cleavage sites from the first plurality. As demonstrated in the examples, the efficiency of size-based separation of polynucleotide fragments depends on the method that is used for this separation. When the size difference between an insert and the fragments is larger, the separation will be easier and/or more complete. When the size difference is smaller, separation can still be performed, but might lose efficiency, for example leading to incomplete separation.

The fragments can have any length and any distribution of lengths. For practical purposes, it is preferred to divide the backbone into fragments having a length of at most 1000 bp, to allow easier separation from relatively small inserts. In preferred embodiments is provided the backbone according to the invention, wherein the fragments have a length of at most 900, 800, 700, 650, 600, 550, 500, 450, 400, 350, 300, 250, 200, 150, or 100 bp or less, preferably of at most 700, 650, 600, 550, 500, 450, 400, 350, 300, 250, 200, 150, or 100 bp or less, more preferably of at most 550 bp, such as of about 500 bp or less. Variation in length by single nucleotides or by ten or fewer nucleotides is not expected to have a substantial effect on the behavior of a fragment, so fragment size is preferably seen as a target size and not as an absolute barrier. The length of a fragment is preferably defined as the length of the resulting fragment after cleavage has been performed. For practival purposes, it is preferred to divide the backbone into fragments having a length of at least 20, 30, 40, 50, 60, 70, 80, 90, 100, 110, 120, 130, 140, 150, 160, 170, 180, 190, 200, 210, 220, 230, 240, or 250 bp, more preferably at least about 100 bp, even more preferably at least about 150 bp, most preferably at least about 200 bp.

In other preferred embodiments, the fragments have a length of at most 40%, 35%, 30%, 25%, 20%, 15%, 10%, or 5% of the length of the insert. For a backbone without an insert, or for an empty vector according to the invention, this can mean that the fragments have a length relative to an intended insert. Length in this context relates to the number of nucleotides.

For ease of use, it is preferable to have fragments that are substantially of the same size. This allows the use of more specialized separation techniques. Accordingly, in preferred embodiments, the fragments have a length distribution wherein the shortest fragment has at least 20%, 25%, 30%, 35%, 40%, 45%, 50%, 55%, 60%, 65%, 70%, 75%, 80%, 85%, 90%, 95%, or 98% of the length of the longest fragment. Of course the insert is not to be considered a fragment, despite its release during fragmentation. In other preferred embodiments, the length difference between the shortest and the longest fragment is no more than 400, 350, 300, 250, 200, 150, 100, 50, or 20 bp, preferably no more than 250 bp.

The application of cleavage means to cleave the first plurality of cleavage sites, leading to fragments, is referred to herein as degradation, immolation, shredding, or cleavage, as will be apparent from context. A polynucleotide sequence that is divided into fragments is referred to herein as a self-immolative sequence, or a degradation-ready sequence, or a sequence primed for degradation.

### Possible Functional Elements of the Backbone or of the Insert

The backbone according to the invention and an insert for use in the invention can comprise different polynucleotide elements that each serve a particular function; these are referred to as functional elements. Examples of functional elements are markers, multiple cloning sites, expression enhancing elements, origins of replication, and promoters. Backbone design and vector design are an established field of art, and a skilled person will know how to design a backbone or insert for a particular application.

### Marker

In preferred embodiments a backbone according to the invention or an insert for use in the invention comprises a polynucleotide encoding a functional marker, preferably a selection marker, wherein the polynucleotide encoding a functional marker is preferably selected from SEQ ID NOs: 14-18, 79, and 110-116, or from a polynucleotide encoding a marker selected from SEQ ID NOs: 69, 70, 81-88; the polynucleotide encoding the functional marker is preferably in the backbone. The marker is preferably derived from SEQ ID NOs: 7-13.

Selection markers are known in the art. A selection marker, also known as a selectable marker, is a gene that when introduced into a cell, especially a bacterium or to cells in culture, confers a trait suitable for artificial selection. Examples of selection markers are resistance markers that confer resistance to for example antibiotics, and biosynthesis markers that complement auxotrophy, or are used for screening, such as LacZalpha. Preferred selection markers are resistance markers, more preferably antibiotic resistance markers. Suitable antibiotic resistance markers are resistance genes against kanamycin, spectinomycin, streptomycin, ampicillin, carbenicillin, bleomycin, erythromycin, polymyxin B, tetracycline, chloramphenicol, and zeocin. In preferred embodiments, the selection marker provides resistance against an antibiotic selected from the group consisting of zeocin (zeo), chloramphenicol (cam), tetracyclin (tet), streptomycin (str), kanamycin (kan), and ampicillin (amp). In more preferred embodiments, this antibiotic is selected from kanamycin (kan) and ampicillin (amp).

The polynucleotide encoding a functional marker is preferably any one of SEQ ID NOs: 14-18, 79, and 110-116. Such a sequence preferably has 70, 75, 80, 85, 90, 91, 92, 93, 94, 95, 96, 97, 98, 99, or 100% sequence identity with the selected SEQ ID NO, more preferably it has 90, 91, 92, 93, 94, 95, 96, 97, 98, 99, or 100% sequence identity, even more preferably it has 95, 96, 97, 98, 99, or 100% sequence identity, most preferably it has 98, 99, or 100% sequence identity, such as 99% or 100% sequence identity. In more preferred embodiments, the polynucleotide encoding a functional marker is selected from any one of SEQ ID NOs: 14-15. A preferred marker is a marker that comprises at least one cleavage site from the first plurality of cleavage sites.

Alternately, the nucleotide encoding a functional marker encodes a functional marker that is preferably derived from any one of Kan, Amp, Zeo, LacZalpha, and chloramphenicol, more preferably from Kan or Amp. The functional marker is preferably derived from any one of SEQ ID NOs: 7-13. It preferably encodes a polypeptide that has 70, 75, 80, 85, 90, 91, 92, 93, 94, 95, 96, 97, 98, 99, or 100% sequence identity with the selected SEQ ID NO, more preferably it has 90, 91, 92, 93, 94, 95, 96, 97, 98, 99, or 100% sequence identity, even more preferably it has 95, 96, 97, 98, 99, or 100% sequence identity, most preferably it has 98, 99, or 100% sequence identity, such as 99% or 100% sequence identity. In more preferred embodiments, a functional marker is derived from any one of SEQ ID NOs: 7-10 or 13, even more preferably from any one of SEQ ID NOs: 7-8. In this context, a marker derived from an amino acid sequence is preferably a polynucleotide encoding that amino acid sequence.

In another aspect the invention provides a polynucleotide encoding a functional marker as defined above, preferably comprising at least 1, 2, 3, or 4 cleavage sites; for polynucleotides wherein not all mutations relative to the wildtype are silent mutations, the encoded polypeptide is also encompassed by the invention.

A marker has no direct utility when it is not functional. Mutation to established markers may decrease their functionality. However, decreased functionality does not equate lack of functionality. For example, decreased functionality may result in more stringent selection when for example an antibiotic resistance marker is less efficient in its function, effectively conferring less resistance. Such an effect is not necessarily detrimental to the backbone—in fact, it is known that less efficient resistance markers can lead to increased copy number of the plasmid, as more selection marker activity is required to allow cell survival. On the other hand, when a self-immolative resistance marker has an activity that is closer to that of the wild type, its will be easier to use that resistance marker in existing protocols because its behavior will be more predictable. Accordingly, the level of functionality of a selection marker is dependent on the intended use of the backbone. In preferred embodiments, the activity of a self-immolative resistance marker is at least 30, 40, 50, 60, 70, 80, 90, 95, 99, or 100% of that of wild type resistance marker, more preferably at least 70%, even more preferably at least 90%.

Vectors with a marker, or elements for activity selection of a specific sequence, can be easily selected after transfection of the vector. Vectors without a marker can have increase utility in gene therapy, for example as described in WO9605297.

### Multiple Cloning Site

A multiple cloning site (MCS) is a sequence in a backbone harbouring a number of recognition sites for restriction enzymes to facilitate cloning of fragments in that vector. This sequence is commonly small (<100 bp) and the recognition site sequences are generally uniquely present in the multiple cloning site, and not found in the rest of the backbone or the fragment of interest to be cloned (the insert). Multiple coning sites are commonly known in the art. Preferred MCS have a length of at most 300, 250, 200, 190, 180, 170, 160, 150, 140, 130, 120, 110, 100, 90, 80, 70, or fewer bp. Preferred MCS comprise about 4-30, 6-25, 8-20, 10-15 restriction sites.

In preferred embodiments is provided the vector backbone according to the invention, wherein the vector backbone comprises a polynucleotide encoding a multiple cloning site. An MCS can also be in an insert for use in the invention; this can be advantageous when amplification of the polynucleotide encoding the MCS is required. Preferably, a multiple cloning site does not comprise a cleavage site of the first plurality of cleavage sites, and/or comprises at least one restriction site that is not in the first plurality of cleavage sites. This allows use of the MCS without triggering immolation of the backbone. A preferred MCS comprises a BssHII restriction site. BssHII is compatible with MluI which is a preferred cleavage means for triggering immolation. Conversely, a cleavage site present in an MCS can be used to help divide the backbone into smaller fragments, when a restriction enzyme recognizing that cleavage site is used as additional cleavage means for immolation. Preferably, the MCS comprises restriction sites for restriction enzymes that can be used under the same reaction conditions as the cleavage means. This allows exact liberation of the insert simultaneous with immolation, which improves control over the insert that can be obtained after immolation.

In preferred embodiments, the backbone according to the invention comprises a polynucleotide encoding an MCS that shares at least 80%, more preferably at least 90%, most preferably at least 98% or 100% sequence identity with SEQ ID NO: 19. More preferably, the backbone according to the invention comprises a polynucleotide encoding an MCS that comprises the same restriction sites as SEQ ID NO: 19. In preferred embodiments, the backbone according to the invention comprises a polynucleotide encoding an MCS with recognition sites for restriction enzymes as described above, preferably for restriction enzymes that recognise six or seven nucleotides. It is preferred when recognition sites are for enzymes that result in the same overhang as any restriction enzymes in the first plurality of cleavage means. An example is BssHII, MauBI and AscI when MluI is in the first plurality.

A backbone can have an MCS when it is to be used with any type of insert, in which case the MCS allows tailored selection of a restriction enzyme for insertion of an insert into the plasmid. A backbone that is designed for use with only a single insert, or with an insert that is always inserted using the same restriction enzyme, does not need an MCS.

### Promoter

In preferred embodiments the backbone according to the invention or the insert for use in the invention comprises a promoter, preferably a promoter that is configured to be operably linked to a sequence of interest in the insert, or to a nucleotide sequence encoding a marker as defined earlier herein. Accordingly, the promoter can be configured to be operably linked to a sequence that is not present in the backbone itself, when no insert is present. Promoter refers to a nucleic acid sequence, located upstream or 5′ to a translational start codon of an open reading frame (or protein-coding region) of a gene or to a nucleic acid sequence that functions to control the transcription of DNA, and that is involved in recognition and binding of RNA polymerase II and other proteins (trans-acting transcription factors) to initiate transcription. The term promoter refers to a nucleic acid fragment that functions to control the transcription of one or more nucleic acid sequences, located upstream with respect to the direction of transcription of the transcription initiation site of the sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one skilled in the art to act directly or indirectly to regulate the amount of transcription from the promoter. The promoter does not include the transcription start site (TSS) but rather ends at nucleotide −1 of the transcription site, and does not include nucleotide sequences that become untranslated regions in the transcribed mRNA such as the 5′-UTR. Promoters suitable for use in a backbone according to the invention may be tissue-specific, tissue-preferred, cell-type specific, inducible and constitutive promoters. Tissue-specific promoters are promoters which initiate transcription only in certain tissues or within certain cells of that tissue. Expression in a tissue-specific manner may be only in individual tissues or in combinations of tissues. Tissue-preferred promoters are promoters that preferentially initiate transcription in certain tissues. Cell-type-specific promoters are promoters that primarily drive expression in certain cell types. Inducible promoters are promoters that are capable of activating transcription of one or more DNA sequences in response to an inducer. The DNA sequences will not be transcribed when the inducer is absent or when an inhibitor is present, such as for the Tet-off promoter. Activation of an inducible promoter is established by application of the inducer or by absence of the inhibitor. Constitutive promoters are promoters that are active under many environmental conditions and in many different tissue types. Preferably, capability to initiate transcription is established in an expression system using an expression construct comprising said promoter operably linked to a nucleotide sequence of interest using a suitable assay such a RT-PCR or Northern blotting. A promoter is said to be capable to start transcription if a transcript can be detected or if an increase in a transcript level is found of at least 5%, 10%, 15%, 20%, 30%, 40%, 50%, 60%, 70%, 80%, 90%, 100%, 200%, 300%, 500%, 1000%, 1500%, 2000%, 4000%, 8000%, 16000%, or more as compared to transcription using a construct which only differs in that it is free of said promoter. In a further preferred embodiment, capability to initiate expression is established in an expression system using an expression construct comprising said promoter operably linked to a nucleotide sequence encoding a protein or polypeptide of interest. Preferably, said protein or polypeptide of interest is a secreted protein or polypeptide and expression of said protein or polypeptide of interest is detected by a suitable assay such as an ELISA assay, Western blotting or, dependent on the identity of the protein or polypeptide of interest, any suitable protein identification and/or quantification assay known to the person skilled in the art. Absent expression-suppressing factors, a promoter is said to be capable to initiate expression if the protein or polypeptide of interest can be detected or if an increase in an expression level is found of at least 5%, 10%, 15%, 20%, 30%, 40%, 50%, 60%, 70%, 80%, 90%, 100%, 200%, 300%, 500%, 1000%, 1500%, 2000%, 4000%, 6000%, or 10000% as compared to expression using a construct which only differs in that it is free of said promoter. A promoter is preferably configured to be operably linked to a polynucleotide sequence comprised in the insert.

Examples of promoters suitable for use in a backbone according to the invention are constitutive promoters or inducible promoters, preferably inducible promoters. In preferred embodiments the backbone according to the invention of the insert for use in the invention comprises a polynucleotide sequence encoding Lac promoters, trp, tac, Bla promoter, chloramphenicol resistance promoter (Pcat), phage promoters such as Lambda promoters P1, P5, P22 and Pr, P2, pPrpB, pTetO, and hybrid promoters such as lac-trp, Tac (lactose/IPTG inducible), alcB, T7, LacO, araBAD, pBAD (arabinose inducible, glucose repressible), pTrc, hybrid of trp and lac promoters (Lactose/IPTG inducible), bacillus subtilus promoters, Pspac, SacB system promoters, P43, M13K07 genes 1-8 promoters, promoters active in mammalian cells such as CMV, SV40, EF1a, CAG (Eukaryotic promoter database (EPD), available at epd.vital-it.ch/index.php); other promoters such as UAS, PolII (H1/U6), MT, unc-54, Polyhedrin, SP6, TMV, plant promoters used in plants such as CaMV, 35S, Ubiquitin, Actin, Lat52, PR1a, PR2d, synthetic plant promoters (W. Liu and C. N. Steward, Current Opinion in Biotechnology 2016, 37:36-44), promoters active in yeast such as Gal4, AOX, PGK, ADH1, ADE2, TRP1.

Preferred promoters can be used to clone, identify and/or isolate fragments of interest. They include promoters involved in expression of markers as well as promoters involved of expression of gene sequence of the sequence of interest. Promoters include viral, archaeal, prokaryotic and eukaryotic promoters. Preferably bacterial, fungal, plant, insect or animal promoters, more preferably promoters known in the art used for cloning fragments of interest, like Lac promoters, or promoter useful for activity cloning or expression cloning (viral, archaeal, prokaryotic, eukaryotic).

More preferred promoters are Bla, T7, Pcat, pBAD, hybrid promoters, SP6, AOX, ADH, GAL, TRP1, CMV EF1a, and SV40; most preferred promoters are Bla, T7, Pcat, hybrid promoters, SP6, AOX, ADH, GAL, CMV, and SV40. A highly preferred promoter is a promoter that comprises at least one cleavage site from the first plurality of cleavage sites.

Vectors with a promoter that is configured to be operably linked to an insert can readily express a polynucleotide sequence encoded on that insert. Vectors that are intended for use solely in insert amplification have no use for a promoter and are more efficiently amplified when no such excess sequence is present.

### Origin of Replication

An origin of replication (also called the replication origin, or Ori) is a particular sequence in a genome, a plasmid, or a vector at which replication is initiated. This can either involve the replication of DNA in living organisms such as prokaryotes and eukaryotes, or that of DNA or RNA in viruses, such as double-stranded RNA viruses. Oris can vary between organisms. They commonly feature a distinctive region containing a higher than average number of adenine and thymine residues (the AT-rich region) where, during the process of replication initiation, the initial destabilization (opening) of the double helix takes place.

In preferred embodiments, the backbone according to the invention comprises a polynucleotide that encodes a functional Ori, preferably wherein the Ori comprises at least one cleavage site from the first plurality of cleavage sites, or wherein preferably the Ori is immediately preceded and followed by a cleavage site from the first plurality of cleavage sites. This allows fragmentation of the Ori, or its immolation into a distinct fragment that is sufficiently small to allow practice of the invention. Accordingly, when the Ori has a length more than the desired size of degraded fragments, such as more than 1000 bp, it should contain a cleavage site from the first plurality; if not, it can be preceded and followed by such a cleavage site to allow efficient fragmentation of the backbone comprising such an Ori.

A functional Ori as used herein is an Ori that allows replication of a vector it is comprised in. Functionality of an Ori is preferably expressed as a percentage, wherein the percentage represents the relative copy number at which a vector is maintained when comprising a mutated Ori as compared to the copy number of a vector comprising that Ori it is derived from, such as the wildtype Ori, or such as the Ori as it is known in the art. Preferably, a functional Ori allows a plasmid to be maintained at a copy number that is at least about 0.01% of the copy number that would be achieved with the Ori it is derived from, preferably under conditions as described in the examples. More preferably, the copy number is at least 0.1%, 1%, 5%, 10%, 25%, 50%, 75%, 80%, 85%, 90%, 95%, or 100%. Even more preferably, the copy number is at least 5%, 10%, 25%, 50%, 75%, 80%, 85%, 90%, 95%, or 100%; still more preferably it is at least 10%, 25%, 50%, 75%, 80%, 85%, 90%, 95%, or 100%. In preferred embodiments, the copy number in pSC is at least 3, 4, or 5, more preferably at least 5. In preferred embodiments, the copy number in pMB is at least 10, 11, 12, 13, 14, or 15, more preferably at least 15. In preferred embodiments, the copy number in pUC is at least 350, 400, 450, or 500, more preferably at least 500. In preferred embodiments, the copy number in E. coli is at least 3, 4, or 5, more preferably at least 5; this is preferable for backbones derived from pSC. In preferred embodiments, the copy number in E. coli is at least 10, 11, 12, 13, 14, or 15, more preferably at least 15; this is preferable for backbones derived from pMB. In preferred embodiments, the copy number in E. coli is at least 350, 400, 450, or 500, more preferably at least 500; this is preferable for backbones derived from pUC.

The polynucleotide encoding a functional Ori is preferably derived from a yeast, bacterial, or viral Ori, such as from any one of SEQ ID NOs: 20-26. Such a sequence preferably has 70, 75, 80, 85, 90, 91, 92, 93, 94, 95, 96, 97, 98, 99, or 100% sequence identity with the selected SEQ ID NO, more preferably it has 90, 91, 92, 93, 94, 95, 96, 97, 98, 99, or 100% sequence identity, even more preferably it has 95, 96, 97, 98, 99, or 100% sequence identity, most preferably it has 98, 99, or 100% sequence identity, such as 99% or 100% sequence identity. In more preferred embodiments, the polynucleotide encoding a functional Ori has at least 70, 75, 80, 85, 90, 91, 92, 93, 94, 95, 96, 97, 98, 99, or 100% sequence identity with any one of SEQ ID NOs: 27-29, even more preferably from any one of SEQ ID NOs: 27-28. This sequence identity is preferably at least 90%, more preferably at least 95%, still more preferably at least 98%, most preferably 99% or 100%, such as 100%.

A backbone with an Ori is useful for amplification of the backbone or of a vector comprising a backbone and an insert. A backbone without an Ori is useful in gene therapy, or when an Ori is encoded by the (intended) fragment of interest, or in case of preparative PCR.

### Expression Enhancing Elements

Specific additional polynucleotide sequences can increase the utility of a backbone according to the invention, or of an insert for use in the invention; particularly, certain polynucleotide sequences can enhance the expression of polypeptides or RNAs encoded by vectors comprising a backbone according to the invention, or of systems into which inserts for use in the invention are inserted after their isolation from a self-immolative backbone. These enhancing polynucleotide sequences are referred to herein as expression enhancing polynucleotides. In preferred embodiments is provided a recombinant polynucleotide vector according to the invention, wherein the backbone according to the invention or preferably the polynucleotide insert (that is: the polynucleotide for use in the invention) comprises an expression enhancing polynucleotide having at least 70% sequence identity with any one of SEQ ID NOs: 30-67. In more preferred embodiments, the expression enhancing polynucleotide has at least 75, 80, 85, 90, 91, 92, 93, 94, 95, 96, 97, 98, 99, or 100% sequence identity with any one of SEQ ID NOs: 30-35, 37, 51-60, 63-65, and 67, even more preferably at least 90%, more preferably still at least 95%, most preferably at least 98%, such as 100%. A preferred expression enhancing element is an expression enhancing element that comprises no cleavage site from the first plurality of cleavage sites.

Examples of expression enhancing polynucleotides are known in the art. For example, EP1432808 discloses SEQ ID NO: 30, which enhances expression of sequences of interest of which it is comprised in the 5′UTR. Another example is WO2012044171, which discloses SEQ ID NO: 31, which enhances expression of sequences of interest of which it is comprised in the 5′UTR. Another example is WO2015102487, which discloses nucleic acid sequences encoding a first promoter, a second promoter, and an optional nucleotide sequence of interest, wherein said first promoter and second promoters are configured to be both operably linked to said optional nucleotide sequence of interest, and wherein said second promoter is an intronic promoter, flanked by a first intronic sequence located upstream of said promoter and a second intronic sequence located downstream of said promoter. Exemplary embodiments of such dual promoter constructs featuring both a conventional promoter and an intronic promoter are represented by SEQ ID NOs: 55-58. Preferred examples of further expression enhancing polynucleotides are SEQ ID NOs: 59-67.

### Selection and Design of Functional Elements

Functional elements as described herein often comprise a cleavage site from the first plurality of cleavage sites. This is because the polynucleotides encoding the functional elements can have a size that is larger than the intended size of the fragments. To introduce a cleavage site into a polynucleotide that encodes a polypeptide, it is most convenient to analyze the sequence of the functional element to find sequences that already have high sequence identity with the sequence of an intended cleavage site. For example when it is intended to introduce the cleavage site GAATTC (the restriction recognition site for the restriction endonuclease EcoRI) in a polynucleotide, it is convenient to analyze the polynucleotide for sequences of six nucleotides that already share 3, 4, 5, or all 6 of those nucleotides; preferably that already share 5 or 6 of those nucleotides. When 6 nucleotides are shared, no mutations are required. When 5 nucleotides are shared, only a single mutation is required. For such screening it is convenient to use freely available online tools, such as REBASE (Roberts et al., Nucleic Acids Research, 43 D1, 2015, D298-D299, DOI: 10.1093/nar/gku1046), where nucleotide sequences can be compared (blasted) against known restriction enzyme recognition sites.

Conversely, when analysis of a polynucleotide sequence reveals a sequence known to be a possible cleavage site, it can be convenient to consider this cleavage site to be a cleavage site of the first plurality. Use of its associated cleavage means will contribute to fragmentation of the functional element comprising said cleavage site. For example, when a given restriction recognition site is natively present in a marker, introduction of this restriction recognition site in other regions of the backbone according to the invention can facilitate backbone design. Screening of polynucleotide sequences can be done using freely available online tools, such as NEBcutter (Vincze et al., Nucleic Acids Res. 31: 3688-3691 (2003)).

It is preferable to introduce a silent mutation that introduces the cleavage site. A silent mutation is a mutation in a polynucleotide that does not alter the encoded polypeptide. Silent mutations are known in the art, and can be made based on the known codon tables.

When no silent mutations can be made, conservative mutations can be made wherein an encoded amino acid residue is mutated to a similar amino acid residue. Conservative mutations are known in the art, and for example preserve residue charge, polarity, or size. Examples of conservative mutations are Arg to Lys, Glu to Asp, Asn to Gln, Gly to Ala, Ser to Thr, Leu to lie, etc. Non-silent mutations are preferably made in unordered regions of the encoded polypeptide, such as in random coils or close to a terminus such as the N-terminus.

When neither silent nor conservative mutations can be made to introduce a cleavage site, a non-conservative mutation or an insertion of an amino acid residue can be made. Preferred amino acid residues for insertion are small, uncharged amino acid residues such as Ala, Gly, Ser, or Thr, preferably Ala, Ser, or Thr, more preferably Thr. For example, the dipeptide Thr-Arg can be encoded by ACGCGT, which is a recognition site for the MluI restriction endonuclease. Thus, when an encoded polypeptide comprises Arg, insertion of Thr can lead to insertion of a cleavage site, namely the MluI restriction site. Similarly, when an encoded polypeptide comprises Thr, insertion of Arg can lead to insertion of a cleavage site. Insertion of Thr is preferred because it is known in the art that insertion of small, uncharged residues is less likely to disrupt the functionality of a polypeptide. The effect of a mutation, when not a silent mutation, can be assessed using any method known in the art. Examples of mutation planning and assessment are for example given by Morrison and Weiss (2001) “Combinatorial alanine-scanning” Curr Opin Chem Biol 5(3): 302-7, DOI: 10.1016/S1367-5931(00)00206-4.

When a cleavage site is to be introduced in a functional element that does not encode a polypeptide, it is preferred to introduce a cleavage site through a mutation that preserves known sequence motifs. For example, a mutation in an AT-rich region preferably introduces either T or A. For example, when a mutation known to be in the stem of a stem-loop region is introduced, it is preferably accompanied by a complementarity-preserving mutation in the base pairing residue at the other arm of the stem.

A skilled person knows how to introduce specific mutations in a polynucleotide, and polynucleotides with custom sequences are commercially available from various contract suppliers. Once a self-immolative functional element or a backbone according to the invention has been designed, it can be tested for functionality using routine techniques. For example, a backbone comprising a selection marker can be subjected to the associated selection pressure. Alternatively, a self-immolative backbone can be equipped with an insert encoding a reporter polypeptide operably linked to a promoter. In such a case, the expression of the reporter polypeptide is correlated to backbone functionality. Examples of reporter polypeptides are fluorescent proteins such as GFP or DsRed, and enzymes that can be easily assayed such as alkaline phosphatase, secreted alkaline phosphatase (SeAP), or luciferase.

In preferred embodiments a backbone according to the invention or an insert for use in the invention comprises a polynucleotide encoding an origin of replication, a polynucleotide encoding a promoter, a polynucleotide encoding a functional marker, an expression enhancing polynucleotide, and a polynucleotide encoding a multiple cloning site; more preferably the origin of replication, the multiple cloning site, and the functional marker are comprised in the backbone according to the invention, and the expression enhancing nucleotide and optionally the promoter are more preferably comprised in the insert for use in the invention.

In preferred embodiments a backbone according to the invention or an insert for use in the invention comprises a polynucleotide encoding an origin of replication, a polynucleotide encoding a functional marker, an expression enhancing polynucleotide, and a polynucleotide encoding a multiple cloning site; more preferably the origin of replication, the multiple cloning site, and the functional marker are comprised in the backbone according to the invention, and the expression enhancing nucleotide is more preferably comprised in the insert for use in the invention.

In preferred embodiments a backbone according to the invention or an insert for use in the invention comprises a polynucleotide encoding an origin of replication, a polynucleotide encoding a functional marker, and a polynucleotide encoding a multiple cloning site; more preferably the origin of replication, the multiple cloning site, and the functional marker are comprised in the backbone according to the invention.

In preferred embodiments a backbone according to the invention or an insert for use in the invention comprises a polynucleotide encoding an origin of replication, and a polynucleotide encoding a functional marker; more preferably the origin of replication and the functional marker are comprised in the backbone according to the invention.

In preferred embodiments a backbone according to the invention or an insert for use in the invention comprises a polynucleotide encoding an origin of replication, a polynucleotide encoding a functional marker, and a polynucleotide encoding a multiple cloning site, wherein the cleavage sites are endonuclease recognition sites; more preferably the origin of replication, the multiple cloning site, and the functional marker are comprised in the backbone according to the invention.

In preferred embodiments a backbone according to the invention or an insert for use in the invention comprises a polynucleotide encoding an origin of replication, and a polynucleotide encoding a functional marker, wherein the cleavage sites are endonuclease recognition sites; more preferably the origin of replication and the functional marker are comprised in the backbone according to the invention.

In preferred embodiments a backbone according to the invention or an insert for use in the invention comprises a polynucleotide encoding an origin of replication, a polynucleotide encoding a functional marker, and a polynucleotide encoding a multiple cloning site, wherein the cleavage sites are endonuclease recognition sites, and wherein the fragments have a length of at most 650 bp, preferably of at most 450 bp, more preferably of at most 350 bp, most preferably of at most 200 bp; more preferably the origin of replication, the multiple cloning site, and the functional marker are comprised in the backbone according to the invention.

In preferred embodiments a backbone according to the invention or an insert for use in the invention comprises a polynucleotide encoding an origin of replication, and a polynucleotide encoding a functional marker, wherein the cleavage sites are endonuclease recognition sites, and wherein the fragments have a length of at most 650 bp, preferably of at most 450 bp, more preferably of at most 350 bp, most preferably of at most 200 bp; more preferably the origin of replication and the functional marker are comprised in the backbone according to the invention.

Within this aspect the invention also provides compositions comprising a backbone according to the invention or a vector according to the invention, and further comprising at least one physiologically acceptable excipient. Examples of physiologically acceptable excipients are water, purified water, and aqueous buffers, for example buffers such as described elsewhere herein.

### Method of Using the Backbone

The backbones according to the invention allow efficient isolation of inserts. Methods of using backbones according to the invention therefore focus on this separation. Such methods can be used for efficient preparation of any insert, such as a polynucleotide encoding a polypeptide of interest, or a specific homology-directed repair (HDR) template DNA in CRISPR/Cas mediated DNA-editing, or for preparation of RNA for use in synthetic biology or pharmacy. Such methods can also be used to prepare (circular) RNA for use in e.g. synthetic biology [Programming cells and tissues. New toolkits of biological parts allow powerful cell programming by synthetic biologists DS Glass and U Alon Science (21 Sep. 2018) 361, 1199-1200].

Accordingly, in another aspect, the invention provides a method for separating a polynucleotide insert from a polynucleotide vector backbone, the method comprising the steps of
i) providing a recombinant polynucleotide vector comprising the insert and the vector backbone, wherein the vector backbone comprises a first plurality of cleavage sites that divide the vector backbone into fragments having a length of at most 1000 bp;
ii) contacting the recombinant vector with cleavage means capable of specifically cleaving the first plurality of cleavage sites to produce backbone fragments; and optionally,
iii) separating the insert from the backbone fragments of step ii). Such a method is referred to herein as a method according to the invention. These steps are preferably performed in numerical order.
   Step i)—Provision of a Self-Immolative Vector

The vector, backbone, and insert of step i) are a vector according to the invention, a backbone according to the invention, and an insert for use according to the invention as described elsewhere herein. Accordingly, in preferred embodiments is provided the method according to the invention, wherein the cleavage sites are restriction endonuclease recognition sites. In preferred embodiments is provided the method according to the invention, wherein the insert does not comprise a cleavage site of the first plurality of cleavage sites. Effectively this ensures that when the first plurality of cleavage sites is cleaved, the insert is not cleaved because it has no matching cleavage site that is comprised in the first plurality of cleavage sites. In other words, in preferred embodiments the insert does not comprise a cleavage site that is also comprised in the first plurality of cleavage sites, or does not comprise a cleavage site that also exists in the first plurality of cleavage sites. In preferred embodiments is provided the method according to the invention, wherein the vector backbone further comprises a polynucleotide encoding a functional marker, wherein the polynucleotide encoding a functional marker is preferably selected from SEQ ID NOs: 14-18. In preferred embodiments is provided the method according to the invention, wherein the fragments have a length of at most 900, 800, 700, 650, 600, 550, 500, 450, 400, 350, 300, 250, 200, 150, or 100 bp or less, preferably of at most 550 bp, such as about 500 bp. In preferred embodiments is provided the method according to the invention, wherein the vector backbone comprises a multiple cloning site that does not comprise a cleavage site of the first plurality of cleavage sites. Preferred backbones according to the invention as described herein are preferred for use in a method according to the invention.

Provision of the vector can be from any source. Typically, the vector will be a vector that has been isolated from a culture, wherein the culturing was performed for the purpose of amplifying the vector. In preferred embodiments, the vector is in a composition. In preferred embodiments, the vector has been isolated from a microbial culture, more preferably from a bacterial culture. In preferred embodiments, the vector is in purified water or in a physiologically acceptable aqueous buffer, preferably a buffer that is compatible with the cleavage means of step ii).

Step ii)—Degrading the Backbone

In step ii) the self-immolative vector that was provided in step i) is degraded into its fragments, liberating the insert. This is because in step ii) the recombinant vector is contacted with cleavage means capable of specifically cleaving the first plurality of cleavage sites to produce backbone fragments. Suitable cleavage means have been described earlier herein. In preferred embodiments is provided the method according to the invention, wherein the cleavage means are selected from the group consisting of a restriction enzyme, an RNA-guided DNA endonuclease enzyme, a sequence-specific nuclease, sequence-specific ultrasonication, a sequence-specific oxidative small molecule such as bleomycin, and a sequence-specific hydrolyzing small molecule such as a lanthanide complex; more preferably the cleavage means are selected from the group consisting of a restriction enzyme, an RNA-guided DNA endonuclease enzyme, and a sequence-specific nuclease; even more preferably the cleavage means are selected from the group consisting of a restriction enzyme and an RNA-guided DNA endonuclease enzyme, most preferably a restriction enzyme.

The contacting of step ii) amounts to using the cleavage means according to its known use. Commonly, the contacting entails the addition of the vector of step i) to a suitable reaction buffer comprising an effective amount of the cleavage means; conversely an effective amount of the cleavage means can be added to a suitable reaction buffer comprising the vector of step i). Suitable buffers depend on the cleavage means and are well known in the art. For example, when the cleave means comprise restriction enzymes, a suitable buffer can be CutSmart (registered trademark) buffer from New England BioLabs (NEB), which comprises 50 mM potassium acetate, 20 mM tris-acetate, 10 mM magnesium acetate, 100 μg/ml BSA, pH 7.9 at 25° C.; another suitable buffer is restriction enzyme buffer SH from Sigma-Aldrich (PubChem Substance ID 24891729); other suitable buffers are described in Khan et al., cited earlier herein. The use of cleavage means per se to cleave a polynucleotide is broadly known in the art, and a skilled person can select suitable conditions to perform the cleavage.

The cleavage means specifically cleave the cleavage sites of the first plurality. This means that only the cleavage sites of the first plurality are cleaved, which degrades the vector into its constituent fragments, resulting in a mixture comprising said fragments and further comprising the insert. Because the insert is devoid of cleavage sites of the first plurality, it is not cleaved into smaller fragments, allowing easy separation of the insert from the smaller backbone fragments. Because the cleavage means are specific for the cleavage sites of the first plurality, no other polynucleotide products are produced besides the fragments and the insert.

In preferred embodiments is provided the method according to the invention, wherein the cleavage means comprise 3, 2, or 1 species of restriction enzymes, preferably wherein the restriction enzyme recognizes a restriction site of 6 or 7 nucleotides, more preferably selected from the group consisting of BstZ17I and MluI. More preferred embodiments provide the method according to the invention, wherein the first plurality of cleavage sites comprises only restriction sites, and comprises no more than 6, 5, 4, 3, 2, or 1, preferably no more than 3, 2, or 1, more preferably no more than 2 or 1, most preferably comprises only 1, species of restriction site, wherein the restriction sites are preferably selected from the group consisting of restriction sites for restriction enzymes as defined earlier herein, most preferably selected from restriction sites for BstZ17I and MluI.

Step iii)—Isolation of the Insert

Step iii) is an optional step wherein the insert is isolated, that is wherein it is separated from the backbone fragments of step ii). In preferred embodiments of the method according to the invention, step iii) is included. An advantage of the invention is that the insert has a size that is larger, often substantially larger, than that of any other polynucleotide present in the mixture from which the insert is to be isolated. In conventional insert isolation the insert is often smaller than the opened vector backbone, or both molecules can have a similar size. This means that for conventional insert isolation gel extraction is often the most likely choice of separation technique.

In molecular biology, “gel extraction” or gel isolation is a technique used to isolate a desired fragment of intact DNA from an agarose gel following agarose gel electrophoresis. An example of gel extraction is described in the examples, where it is used as a reference technique. Gel extraction is laborious, requires manual dexterity, and is prone to contaminate the isolated insert with for example chemical contaminants, as described in the example. In preferred methods according to the invention, step iii) does not comprise gel extraction, or does not comprise preparative gel electrophoresis. In this context, preparative gel electrophoresis is electrophoresis that is performed with the intent of later isolating a fraction from the gel.

In preferred embodiments is provided the method according to the invention, wherein the separation of step iii) uses a technique selected from a spin column, a size exclusion column, and solid phase reversible immobilization (SPRI). Preferred separation techniques are a spin column, a size exclusion column, and solid phase reversible immobilization (SPRI); a spin column is most preferred, for its convenience.

The size difference between the insert and the fragments allows convenient separation using size exclusion techniques. Size exclusion techniques are known in the art, for example as described by Potschka (Macromolecules, 1991, 24(18), pp 5023-5039, DOI: 10.1021/ma00018a008). Size exclusion techniques generally use a column material. The smaller fragments can be captured in or by the column material, while the larger insert elutes with a low retention time. Inversely, the smaller fragments can elute in void volume while the larger insert is captured in or by the column material owing to its size. This latter technique can also be seen as a solid phase extraction, or solid phase reversible immobilization (SPRI), because it relies on the fact that the larger insert binds to the column material, often silica, owing in part to its size, while the smaller fragments do not bind. Later elution of the insert then allows its isolation. Examples of size exclusion techniques are size exclusion chromatography using a spin column, and size exclusion chromatography using a size exclusion column. A size exclusion column is generally used on a benchtop, and liquids flow through it under gravitational force or under action of a pump. The use of such columns is described in handbooks cited elsewhere herein.

A spin column is generally a disposable miniaturized size exclusion column that can be mounted on a receptacle fit for use in a centrifuge. Once the spin column is filled with sample it can be centrifuged to drive the sample through the size exclusion material. The spin column can then optionally be filled with additional volumes of buffer and centrifuged further times. Spin columns are widely commercially available and are well known, for example as described in Shi et al., PLOS ONE, 2018, DOI: 10.1371/journal.pone.0203011. Spin columns can be columns that retain, such as bind or capture, the fragments, for example in size exclusion material only permeable to small fragments, or due to complementarity to sticky ends of the fragments. Spin columns can also be columns that do not retain the backbone fragments, for example in solid phase reversible immobilization of the insert. Spin columns that retain the fragments and that do not retain the inserts are preferred, particularly spin columns with size exclusion material that captures the fragments.

Alternately the fragments can be removed from the mixture by fragment pulldown based on sticky overhang hybridization. If the cleavage means leads to sticky ends for the fragments it generates, these sticky ends can be used to pull down those fragments. Generally, in such a technique, the fragments associate with a solid-phase carrier complementary to the sticky overhangs. Later removal of the solid-phase carrier thus removes the fragments, leaving the isolated insert in solution.

The separation techniques as described above, and the materials for use in such separation techniques, are known in the art. A skilled person can select an appropriate technique, and can select appropriate materials for use in such a technique. The separation technique of choice preferably provides the insert with a purity of at least 50%, 60%, 70%, 75%, 80%, 85%, 90%, 95%, 96%, 97%, 98%, 99%, 99.5%, 99.6%, 99.7%, 99.8%, 99.9%, or higher, such as 100%. More preferably a purity of at least 95%, 96%, 97%, 98%, 99%, 99.5%, 99.6%, 99.7%, 99.8%, 99.9%, or higher is obtained, even more preferably of at least 99% or higher, most preferably of at least 99.7% or higher.

### Method for Amplifying an Insert

The use of a backbone according to the invention also allows the convenient amplification of an insert. In another aspect the invention provides a method for amplifying a polynucleotide of interest, the method comprising the steps of
i) providing a recombinant polynucleotide vector comprising the polynucleotide of interest as an insert, and a vector backbone according to the invention;
ii) amplifying the recombinant polynucleotide vector of step i) by transforming a suitable microorganism with it and culturing said transformed microorganism in a culture medium under conditions suitable for vector amplification to obtain amplified recombinant polynucleotide vector;
iii) isolating the amplified recombinant polynucleotide vector from the culture medium of step ii) to obtain isolated amplified recombinant polynucleotide vector;
iv) contacting the isolated amplified recombinant polynucleotide vector with cleavage means capable of cleaving the first plurality of cleavage sites to produce backbone fragments; and optionally,
v) separating the nucleotide sequence of interest from the backbone fragments of step iv).

This method is referred to herein as a production method according to the invention. Its steps are preferably performed in numerical order.

In step i) a vector according to the invention is provided, which comprises the polynucleotide of interest as an insert, and which further comprises a backbone according to the invention. Preferably, the vector consists of said insert and said backbone.

In step ii) this vector is transformed into a suitable microorganism. In preferred embodiments the recombinant vector is transected to a suitable microorganism. A skilled person is well aware of which microorganisms can be used for vector amplification, such as E. coli or a suitable yeast or fungus—numerous handbooks on the topic exist, such as Sambrook et al., Molecular cloning: a laboratory manual. New York: Cold spring harbor laboratory press, 1989; or Green and Sambrook, 2012, ISBN 978-1-936113-42-2. Similarly, transformation and transfection are known in the art, and can be performed in any way a skilled person sees fit, for example such as described in Sambrook et al. Examples of suitable transfection methods are transfection using transfection lipids such as those described in Damen et al., (MedChemComm 2018, 9(9):1404-1425, DOI: 10.1039/c8md00249e), using transfection polymers such as described in Zhang and Wagner (Top. Curr. Chem. 2017, 375(2):26, DOI: 10.1007/s41061-017-0112-0), electroporation, transformation using competent or supercompetent cells, microinjection, gene bombardment, et cetera. After this transformation the transformed microorganism comprising the vector is cultured to amplify the vector. Again, such steps are broadly known in the art and a skilled person can select the appropriate steps by routine selection, optionally guided by the sources cited above.

In step iii) the amplified vector is isolated. The amplified vector comprises the insert, which has been amplified along with the vector as it is therein comprised. Isolation of a vector from a microorganism is a standard procedure that is commonly known, and can be performed in any way a skilled person sees fit, optionally guided by the sources cited above, or by the examples described later herein. Suitable methods are plasmid preparation such as miniprepration, midipreparation, or maxipreparation, for example as described by Birnboim and Doly (1979) Nucleic Acids Res. 7(6): 1513-23, DOI: 10.1093/nar/7.6.1513.

In step iv) of the production method according to the invention the isolated vector according to the invention is degraded or immolated, similar to step ii) of the method according to the invention. Features and definitions as provided there are also applicable for step iv) of the production method according to the invention.

In step v) of the production method according to the invention, which is an optional step, the insert is isolated, that is it is separated from the backbone fragments of step iv). In preferred production methods according to the invention, step v) is included. This step is substantially similar to step iii) of the method according to the invention. Features and definitions as provided there are also applicable for step iv) of the production method according to the invention.

### Use of the Backbone or Vector

The backbone according to the invention and the vector according to the invention can be used in methods according to the invention, or in production methods according to the invention. Accordingly, in a further aspect the invention provides use of a polynucleotide vector backbone according to the invention, or of a recombinant polynucleotide vector according to the invention, for the purification of a polynucleotide insert.

The invention also provides use of a cleavage means for converting a polynucleotide vector comprising an insert and a vector backbone into:
fragments having a length of at most 1000 bp; and
the separate insert. Features and definitions have been provided elsewhere herein.
   This use is particularly envisioned for cleavage means such as RNA-guided DNA endonuclease enzymes, which can be configured to recognize any particular sequence as a cleavage site.

In preferred embodiments the backbone according to the invention is used in a method for enhancing transcription of a nucleotide sequence of interest in a eukaryotic cell, the method comprising the steps of:
i) providing a recombinant polynucleotide vector comprising the polynucleotide of interest as an insert, and a vector backbone according to the invention;
ii) amplifying the recombinant polynucleotide vector of step i) by transfecting it to a suitable microorganism and culturing said microorganism in a culture medium under conditions suitable for vector amplification to obtain amplified recombinant polynucleotide vector;
iii) isolating the amplified recombinant polynucleotide vector from the culture medium of step ii) to obtain isolated amplified recombinant polynucleotide vector;
iv) contacting the isolated amplified recombinant polynucleotide vector with cleavage means capable of cleaving the first plurality of cleavage sites to produce backbone fragments;
v) separating the nucleotide sequence of interest from the backbone fragments of step iv) to obtain an isolated nucleotide sequence of interest;
vi) integrating the isolated nucleotide sequence of interest in the genome of a eukaryotic cell, preferably a mammalian cell or an insect cell, to obtain a transgenic cell;
vii) culturing the transgenic cell under conditions conducive to expression of the nucleotide sequence of interest.

For steps i) through v) features and definitions as described above apply. In step vi) the isolated nucleotide sequence of interest is integrated into the genome of a eukaryotic cell to obtain a transgenic cell. This can be done via any means known in the art, such as via lipid transfection or electroporation, more preferably such as described in Example 4.3. In preferred embodiments a pool of transgenic cells is obtained. In step vii) the transgenic cell, or optionally the pool of transgenic cells, is cultured to express the nucleotide sequence of interest. Preferably, this expression is higher than expression of the amplified recombinant polynucleotide vector of step iii) when it has been not been degraded as in step iv), but has instead been linearized, for example using a restriction enzyme for which only a single restriction site was present in the backbone.

### Kit of Parts

The backbones and vectors according to the invention can be provided as components in a kit of parts. Therefore, in another aspect the invention provides a kit of parts comprising:
i) a polynucleotide vector backbone according to the invention, or a recombinant polynucleotide vector according to the invention; and at least one of
iia) materials for use in a separation technique as defined elsewhere herein, such as spin filters; or
iib) cleavage means as defined elsewhere herein.
   Such a kit is referred to herein as a kit according to the invention.

A kit according to the invention always comprises a polynucleotide vector backbone according to the invention, or a recombinant polynucleotide vector according to the invention. It can have various further parts, described above as iia or iib. In preferred embodiments, both the parts of iia and iib are comprised.

The further parts under iia are materials for use in separating the fragments from the insert. Such materials have been described under step ii) of the method according to the invention.

The further parts under iib are cleavage means, preferably at least all cleavage means required for immolation of the vector or backbone according to the invention. The cleavage means are preferably provided in a suitable separate container. In preferred embodiments the cleavage means are restriction enzymes, and are provided in plastic containers suitable for subzero storage. Suitable containers for each cleavage means are known in the art, and can be selected based on the cleavage means that is to be contained.

### Method for Designing a Self-Immolative Backbone

The invention further relates to a method for designing a backbone according to the invention, the method comprising steps selected from:
i) providing a polynucleotide vector backbone;
ii) screening the provided vector backbone for endonuclease restriction sites;
iii) screening the provided vector backbone for sequences that share 4, 5, 6, or 7 nucleotides with an endonuclease restriction site;
iv-a) implementing at least one silent mutation in the provided vector backbone to convert a sequence of step iii) into an endonuclease restriction site; and/or
iv-b) implementing at least one mutation in the provided vector backbone to convert a sequence of step iii) into an endonuclease restriction site.

Reduction to practice of this method is demonstrated in the Examples. Features and definitions are as described elsewhere herein. Preferably, the method comprises steps i), ii), iii), and at least one of iv-a) and iv-b); more preferably the method comprises steps i), ii), iii), and iv-a). Steps are preferably performed in numerical order.

The silent mutation of step iv-a) is a mutation that alters the oligonucleotide sequence, preferably to introduce an endonuclease restriction site, while not altering the amino acid sequence of an encoded polypeptide, or alternately while not substantially altering the functionality of an encoded polypeptide or of the oligonucleotide sequence. Alteration of functional elements and their functionality is described elsewhere herein.

The mutation in step iv-b) can be the insertion, deletion, or mutation of any residue in the polynucleotide. In preferred embodiments it is the insertion of a codon to introduce an amino acid, or the mutation of one or more nucleotides to alter a codon for one amino acid to a codon for another amino acid. Preferably, this alteration of codons leads to a conservative mutation of amino acids, such as from a charged amino acid to another amino acid with a similar charge, or from a small amino acid to another small amino acid. Conservative mutations are known in the art.

### General Definitions

The term “derived from” in the context of being derived from a particular naturally occurring gene or sequence is defined herein as being chemically synthesized according to a naturally occurring gene or sequence and/or isolated and/or purified from a naturally occurring gene or sequence. A species derived from another species is preferably mutated, in that at least one residue of an amino acid sequence, or of a polynucleotide, or of a polynucleotide encoding the amino acid sequence, is not the same as in the naturally occurring gene or sequence. Techniques for chemical synthesis, isolation and/or purification of nucleic acid molecules are well known in the art. In general, a derived sequence is a partial sequence of the naturally occurring gene or sequence or a fraction of the naturally occurring gene or sequence. Optionally, the derived sequence comprises nucleic acid substitutions or mutations, preferably resulting in a sequence being at least 50%, 55%, 60%, 65%, 70%, 75%, 80%, 85%, 90%, 91%, 92%, 93%, 94%, 95%, 96%, 97%, 98% or 99% identical over its whole length to the naturally occurring gene partial gene or sequence or partial sequence.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value more or less 1% of the value.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components.

In the context of this invention, a decrease or increase of a parameter to be assessed preferably means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

“Sequence identity” or “identity” in the context of amino acid- or nucleic acid-sequence is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. In this document, sequence identity with a particular sequence indicated with a particular SEQ ID NO preferably means sequence identity over the entire length of said particular polypeptide or polynucleotide sequence indicated with said particular SEQ ID NO. However, sequence identity with a particular sequence indicated with a particular SEQ ID NO may also mean that sequence identity is assessed over a part of said SEQ ID NO. A part may mean at least 50%, 60%, 70%, 80%, 90% or 95% of the length of said SEQ ID NO. The sequence information as provided herein should not be so narrowly construed as to require inclusion of erroneously identified bases. The skilled person is capable of identifying such erroneously identified bases and knows how to correct for such errors.

“Identity” can be readily calculated by known methods, including but not limited to those described in Computational Molecular Biology, Lesk, A. M., ed., Oxford University Press, New York, 1988. Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity. Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Preferred program and parameter for assessing identity for nucleic acid comparison is calculated using EMBOSS Needle Nucleotide Alignment algorithm with the following parameters: DNAfull matrix with the following gap penalties: open=10; extend=0.5.

The present invention has been described above with reference to a number of exemplary embodiments. Modifications and alternative implementations of some parts or elements are possible, and are included in the scope of protection as defined in the appended claims. All citations of literature and patent documents are hereby incorporated by reference.

### DESCRIPTION OF DRAWINGS

FIG. 1—Schematic representation of a backbone degradation primed plasmid. The undesired nucleotide sequence, e.g. bacterial backbone, is presented in grey; the fragment of interest is colourless. The backbone harbours regularly distributed cleavage sites such as recognition sites for restriction enzymes (indicated as RE). These sites are not present in the fragment of interest. Cleavage of the cleavage sites triggers degradation of the backbone, allowing one-step quantitative separation of the fragment of interest from the degraded undesired nucleotide sequence.

FIG. 2—Schematic representation of backbone degradation vector without fragment of interest. Functional units kanamycin marker (grey), origin of replication (black) and multiple cloning site (MCS, white) are indicated. The vector from example 1 has a kanamycin resistance gene as marker, and harbours restriction sites for MluI (indicated as RE1) and BstZ17I (indicated as RE2). After degradation of its sequence by restriction digestion the size of the backbone vector fragments ranges from 121 to 342 bp. The sequence of backbone vector of Example 1 is SEQ ID NO: 68.

FIG. 3—Baseline separation of Fragment of interest from backbone fragments by Sephacryl S500HR filtration. Analysis of samples: Lane 1 (I) Input sample; Lane 2 (M) 2 Log DNA ladder; Lane 3 to 28: Fractions. The lanes comprising insert are indicated as “Fragment of interest” and the lanes comprising backbone fragments are indicated as “Vector backbone fragments.”

FIG. 4—Separation of insert from backbone fragments by a ChromaSpin-1000 method. Input is MluI/BstZ17I digested vector (see Example 2). Lane 1: 2 Log marker; Lane 3: 100 μl 1.5 μg/μl Input; Lane 4: Flow-through.

FIG. 5—Separation pattern of spin column material to determine viable ranges of size difference between degraded backbone fragments and insert for a particular spin column. (M): 2 log ladder NEB; (1): Chromaspin1000 eluate of HincII digested λ DNA; (2): HincII digested λ DNA (unpurified).

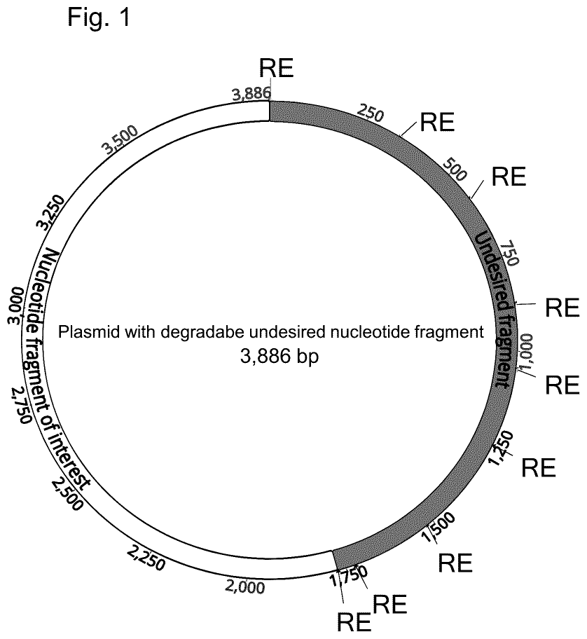

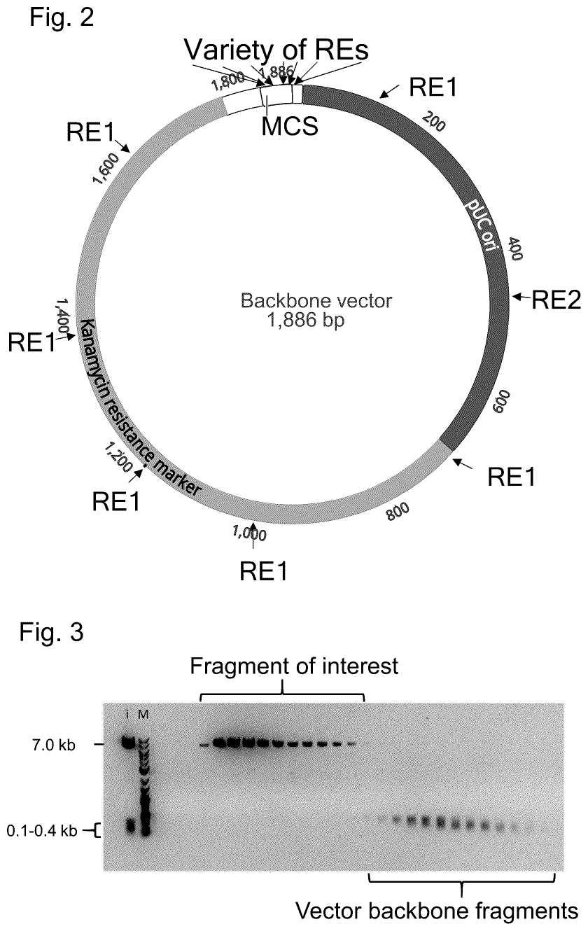

### EXAMPLES

### Example 1—Design and Preparation of a Vector with a Self-Immolative Backbone

### 1.1 Design of a Vector: Introduction of Restriction Enzyme Sites in the Backbone

A design for a vector comprising an insert was made by introducing cleavage sites, in this case restriction sites, for a minimal number of restriction enzymes resulting in fragments smaller than 350 bps. This fragment size allows one-step purification of fragments of interest with a size starting at 1500 bp and higher (see Example 2). The number of enzymes to be used as cleavage means was kept as low as possible to simplify the digestion process and to minimize the number of restriction sites that were to be avoided during synthesis and/or cloning of fragments-of-interest. In this example, two restriction enzymes were selected: MluI and BstZ17I.

While not essential features of all plasmids, typical functional elements of a cloning plasmid are the origin of replication (Ori), a marker, and a multiple cloning site (MCS). Outside of these functional elements, the introduction of restriction sites is relatively easy as no functionality is present. To obtain fragments of maximum 350 bp, introduction of restriction sites is required within the marker and the origin of replication.

### 1.2. Modification of Markers

Polynucleotides encoding for markers can be modified to allow cleavage sites to divide the coding sequence into fragments. In this example the kanamycin and ampicillin selection markers were divided into fragments of at most about 350 bp.

### 1.2.1 Self-Immolative Kanamycin

### In Silico Gene Analysis and Options for Modification

The sequence of the selection marker gene was taken from GenBank JF826242.2. The kanamycin selection marker gene coding for aminoglycoside-3′-phosphotransferase (APH(3′)) and its 816 nt coding sequence are annotated as nt 841-1656 (complement). At the 5′ site of the coding sequence a 130 nt promoter sequence is present. The 145 nt sequence at the 3′ of the coding DNA sequence (CDS) was also considered to be important for gene expression.

Four silent mutations were selected that introduce MluI sites, cutting the CDS into fragments smaller than 200 bp. No silent mutations could be made at the 5′ end of the gene, resulting in a fragment of approximately 430 bp. To allow further digestion within this fragment, inclusion of a cleavage site such as MluI just inside the coding sequence was necessary. The N-terminal 10 amino acid sequence was selected as a target for mutations that would still yield APH(3′) expression and activity. These first 10 amino acids (MSHIQRETSC—SEQ ID NO: 102) were highly promising, because they are unordered (D. Nurizzo et al., The crystal structure of aminoglycoside-3′-phosphotransferase-IIa, an enzyme responsible for antibiotic resistance. J Mol Biol (2003) 327, 491-506) and they apparently do not play a role in the structural function of the enzyme. Moreover, the amino acid sequence at the N-terminal end is not conserved, with different lengths and sequences occurring throughout known variants. Based on this the following two variants were designed: 1) MSHIQTRETSC (SEQ ID NO: 103, first 11 amino acids of SEQ ID NO: 69) with a Thr inserted; the dipeptide TR can be encoded by a MluI recognition site (ACGCGT) and 2) MSHIQRETRSC (SEQ ID NO: 104, first 11 amino acids of SEQ ID NO: 70) with an Arg inserted. These two variants, together with wild-type sequence, were tested for capacity to allow selection on kanamycin.

### Experimental Gene Modifications, Functional and Sequence Analysis

Backbone plasmid was created using five geneblocks (Integrated DNA technologies (IDT), Leuven, Belgium, SEQ ID NOs: 71-75). The geneblocks were dissolved to 0.1 μM in water. Combinations forming a complete backbone (i.e. one variant or wildtype for each of the five fragments were made in 16 μl final reaction volume, containing 0.4 μl of each geneblock at 0.1 μM, 8 μl HiFi assembly mix (NEB E2621), and 6 μl of water (Sigma W4502). Mixtures were incubated 1 hr at 50° C., and stored at −20° C. till further use. Top10 chemically competent cells (Life Technologies C404010) were transformed according the manufacturer's instructions with 2 μl of the assembly reactions. Functionality of the kanamycin resistance gene, i.e. expression and activity of APH(3′), was tested by the ability of the plasmid to confer kanamycin resistance, which is observed by plating the transformants onto LB-Kan plates. Combinations were first made with 1 out of the 5 wildtype fragments replaced with a variant fragment.

In one fragment the four silent mutations in the kanamycin gene were introduced (SEQ ID: 76). Wildtype fragments were used to generate the reference plasmid. Introducing the four silent mutations did not adversely affect kanamycin resistance. Next, either the threonine (SEQ ID NO: 77, encoding the polypeptide of SEQ ID NO: 69) or the arginine insertion (SEQ ID NO: 78, encoding the polypeptide of SEQ ID NO: 70) was introduced to the plasmid with the four silent mutations. The insertion of threonine also resulted in proper transformants, while transformants with an arginine introduced in the N-terminal sequence of APH(3′) were less viable. In subsequent experiments plasmids were used in which the four silent mutations and the threonine-inserted APH(3′) are present.

### 1.2.2 Self-Immolative Ampicillin

The DNA sequence conferring ampicillin resistance to bacteria was redesigned to allow degradation into small fragments. Two silent mutations were introduced into the β-lactamase coding sequence to introduce MluI restriction sites. This lead to a division into fragments of 310, 352, and 524 bp. The resulting selection marker (SEQ ID NO: 79) can be used in backbones according to the invention, for example instead of the kanamycin marker described in Example 1.2.1. To obtain fragments smaller than 400 bp, single amino acid changes by introducing additional MluI and BstZ17I recognition sites were designed, so that the resulting fragments have sizes of at most 352 bp; the resulting selection marker has SEQ ID NO: 15. An example of a vector comprising this selection marker is SEQ ID NO: 80. This redesigned ampicillin resistance gene can be applied for use in a plasmid with a self-immolative backbone. Other suitable self-immolative functional ampicillin variants with one or more altered amino acid were designed and functional: SEQ ID NOs: 81-88. These variants can also be used to generate a self-immolative backbone. One self-immolative ampicillin (SEQ ID NO: 89) was found to not be functional.

### 1.3 Self-Immolative Ori

### In Silico Analysis and Experimental Approach

In the 674 bp region of the origin of replication several mutations were taken into account. The changes selected for were based on the different domains or structures present in the Ori (A. Waugh et al., RNAML: A standard syntax for exchanging RNA information. RNA (2002) 8, 707-717). The initial design consisted of a variety of changes in the 300 bp at the 3′ of the Ori.

Multiple mutations in a conventional Ori derived from pUC (SEQ ID NO: 20) were designed harbouring single nucleotide changes (SEQ ID NO: 90) as well as single nucleotide insertions (SEQ ID NO: 91) at the 3′ of Ori, as well as insertions and nucleotide changes in the center of the Ori (SEQ ID NO: 92) and the stem-loop (SEQ ID NO: 93) which is located between nucleotides 220-310 of the original plasmid backbone sequence (SEQ ID NO: 1). Transformants harboring the mutated Ori were generated as described above. In vivo, the performance of the Ori-modified plasmids was dependent on the exact sequence. For instance, specific mutations at the 3′ of the Ori (see SEQ ID NO: 90) resulted in an Ori whose plasmids yielded transformants with reduced viability, as did the mutations in SEQ ID NOs: 117 and 118.

Successful designs (consisting of changes SEQ ID NO: 94 and SEQ ID NO: 95) and an insert upstream of the Ori (SEQ ID NO: 96) were found using the process described above. They were combined to obtain an Ori with MluI restriction sites that were separated by a maximum of 580 nt. This Ori, in turn, was combined with three new mutations in the Ori bearing the restriction sites for BstZ17I (SEQ ID NO: 97, SEQ ID NO: 98, and SEQ ID NO: 99). SEQ ID NO: 99 was selected for further use and the plasmid harbouring it was fully functional, displaying a copy number and plasmid yield similar to SEQ ID NO: 1. SEQ ID NO: 98 and SEQ ID NO: 97 were also functional.

### 1.4 Self-Immolative Multiple Cloning Site (MCS)

A 52 bp multiple cloning site (MCS) was introduced, including unique recognition sites for SbfI, PstI, NotI, BssHII, SalI and AflII (SEQ ID NO: 19). Owing to its short size, this MCS does not require a cleavage site that is one of the self-immolative cleavage sites.

BssHII is compatible with MluI, so the use of the combination of these three enzymes is beneficial for easy introduction or removal of a fragment of interest via BssHII, and later plasmid degradation via MluI and BstZ17I.

1.5 Assembly into a Vector

A self-immolative vector comprising a resistance marker (SEQ ID NO: 14), an Ori (SEQ ID NO: 27), and a MCS (SEQ ID NO: 19) was assembled (SEQ ID NO: 68) and sequence verified. The plasmid could be selected, had good copy number, and features an MCS allowing insertion of a region of interest. After degradation of its sequence by restriction digestion the size of the backbone vector fragments ranges from 121 to 342 bp (342-241-301-173-211-269-210-121 bp).

### Example 2—Isolating an Insert from Degraded Backbone

2.1 Immolation of a Plasmid with an Insert

Into a plasmid consisting of a self-immolative backbone (SEQ ID NO: 68) an insert was introduced consisting of a secreted alkaline phosphatase (SeAP) coding sequence (SEQ ID NO: 100), preceded by Expression Enhancing Element 1 (SEQ ID NO: 59) and the CMV promoter (SEQ ID NO: 61) and the GS selection marker (SEQ ID NO: 101). This plasmid was treated with MluI and BStZ17I under conditions prescribed by the enzymes' supplier. After digestion, the insert was purified as described below.

### 2.2.1 Size Exclusion Chromatography, Milliliter Scale

Sephacryl S-500 gel filtration column chromatography was carried out to purify the insert from degraded undesired DNA. A HiPrep 16/60 Sephacryl S-500 HR ID16 mm column (GE Healthcare Life Science) mounted on an Akta Pure was rinsed with degassed milliQ water and equilibrated with running buffer (RB=100 mM NaCl, 10 mM Tris/HCl pH=8.5) with a flow of 0.5 ml/min at 37° C. 0.95 ml DNA solution was applied. The column was run with running buffer at a flow of 0.5 ml/min. 2 ml fractions were collected.

Samples were analyzed by gel-electrophoresis (FIG. 3). The insert containing the region of interest (band >7000) and the backbone fragments (121-342 bp) are present in distinct fractions. The method clearly allows a purity level of >99.9%, as fragment of interest is fully separated from backbone fragments.

### 2.2.2 Size Exclusion Chromatography, Microliter Scale (ChromaSpin-1000)

The ChromaSpin-1000 column (Takara/Clontech) was developed for size-dependent separation of DNA. Suppliers' specifications indicate removal of DNA smaller than 420 bp with an efficiency of at least 90% and a removal efficiency of at least 99% efficiency for DNA smaller than 300 bps. The capacity, for 100 μl sample volume and over 90% recovery, is 1 mg/ml.

A ChromaSpin-1000 column was prepared for use by resuspending the matrix by inverting the column ten times, removing bottom and top cap, and placing the column in a collection tube. The column, with the collection tube, was placed in a 14 ml falcon tube and spun (5 min @ 700×g) at room temperature. Columns were placed in fresh collection tubes, 100 μl sample (MluI/BstZ17I digested plasmid (see 2.1) was applied onto the centre of the column matrix and the column with the collection tube, was placed in a 14 ml falcon tube and spun again (5 min @ 700×g) at room temperature. The eluate in the collection tube is the purified insert.

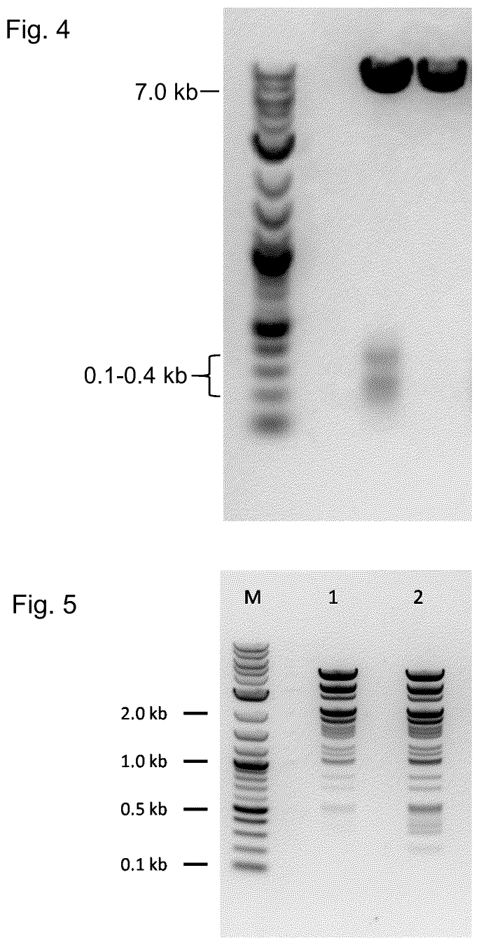

The eluate was analyzed by agarose gel electrophoresis, which indicates that degraded backbone is removed (FIG. 4). Typically, 2-4 mg of plasmid is isolated from 500-600 ml bacterial culture, using Plasmid Maxi Kit (Qiagen). Upon digestion of 100 μl, 1 μg/μl DNA and carrying out clean-up of the insert using the spin column results in 100 μl, 0.4 μg/μl DNA in the filtrate. Thus backbone fragments could be removed after a single non-diluting purification step.

### Example 3—Fragment Size and Insert Size can be Matched to Isolation Techniques

Dependent on the applications, insert sizes range from approximately 1500 (an average size of a protein gene) to 7000-10000 bp (for multimeric protein-specific cassettes and selection marker sequences). A bacterial backbone (including a resistance marker) is approximately 2000 bp. In this example it was studied whether the properties of commercially available spin column material can separate the fragments. Such spin columns are highly convenient for isolating an insert.

DNA from Phage Lambda (NEB) was digested with HincII (NEB). Digested DNA was applied on a spin column (see Example 2) and the eluate was analysed by agarose gel electrophoresis (See FIG. 5). Comparison of fragment pattern before and after spin column separation indicates that fragments smaller than 400-500 kb are removed from the eluate whereas fragments larger than 2000 bp are retained in the eluate. Fragments with a size between 500 and 2000 bp are partially captured by the column material. The values are in accordance with the suppliers' specifications which states that over 90% of >2000 bp fragment will in the eluate. With respect to removing fragment DNA it states that over 90% of fragments smaller than 420 bp and over 99% of fragments smaller than 300 bp will be removed from the sample. This confirms that these particular spin columns are best used to remove undesired fragments with a size of 400 bp or smaller from inserts with a size of 2000 bp or larger.

By using different spin column materials (e.g. Sephacryl S-500 HR (GE Healthcare) in columns (Pierce) or Chromaspin 400) differently sized fragments are separated. For the latter, 90% fragments of smaller than 170 bp are captured by the column, while over 90% of the fragments larger than 950 bp freely pass the column (Chroma Spin Columns User Manual, Clontech, February 2011).

Thus, depending on the preferred size of the insert, a degradable vector backbone may be selected with cleavage sites in the backbone that result in fragments which are removed by choosing the best-fitting separation material, using for example spin columns, sephacryl, or other materials.

### Example 4—Quality Verification of Insert Isolated from Degraded Backbone

### 4.1 DNA Purity Analysis of Agarose-Gel Isolated and Shredded-Spin Column Purified DNA

The invention allows omitting the use of a purification step (agarose purification) which is prone to chemical and biological contamination of isolated DNA. A key chemical impurity in DNA after isolation from agarose gel is guanidinium, which is used to dissolve agarose gel. Its presence can be detected during DNA concentration determination and analysis with nanodrop measurement equipment (OD 230/OD 260/OD280).

DNA was purified using either Chroma Spin columns or agarose gel isolation. Agarose gel isolation consists of the following steps: 1. Running a quantitative amount of DNA on agarose gel; 2. Isolation of the DNA by cutting out the piece of gel harbouring the DNA of interest; 3. Dissolving the agarose by adding 2 volumes of guanidinium containing buffer per volume of agarose gel; 4. Applying the DNA to column material, rinsing the column material twice and eluting the DNA from the column (in two steps).

The use of spin columns is described in the previous examples. Besides not requiring manual dexterity for cutting out a relevant piece of gel, the spin columns only required about 15 minutes of time, where the gel extraction required about 3 hours in total.

In a gel-extraction experiment one time the protocol was followed meticulously, while in a parallel experiment the centrifugation step was different. DNA quality is measured by absorbance ratio's (260:280 nm and 260:230 nm). Isolation from agarose gel provided the following values for isolated inserts:
Experiment 1: A260/A280=1.79-1.80, A260/A230=0.5-0.75;
Experiment 2: A260/A280=1.86-1.86, A260/A230=1.96-2.11.

Only the values of the second experiment meet common specifications (PCR clean-up Gel extraction User manual NucleoSpin® Gel and PCR Clean-up, Machery-Nagel February 2017/Rev. 04). The cause for the deviating values of the first experiment was attributed to the presence of guanidium due to incomplete removal of the agarose solubilizing buffer. The second experiment was carried out meticulously according to protocol.

The data from three representative insert isolation experiments using the backbone-degradation spin column removal method resulted in the following quality levels:
Experiment 1: A260/A280=1.87, A260/A230=2.01;
Experiment 2: A260/A280=1.85, A260/A230=2.05;
Experiment 3: A260/A280=1.88, A260/A230=1.99.

All three experiments resulted in values that meet specifications. Thus it follows that adequate purification from agarose gel depends on properly carrying out protocol, while purification by (spin)column results in DNA which is of at least the same quality as agarose-gel purified DNA, but which is much more robust in variations in protocol.

### 4.2 Endotoxin Contamination Measurements

A relevant biological DNA contaminant is endotoxin. The quality of isolated materials is also determined by the risk of the DNA isolation method to introduce endotoxin. Therefore, in a parallel experiment, a comparison was made between the endotoxin level in DNA samples purified using either agarose gel isolation (see example 4.1) or spin columns (see example 2). The detection was carried out using an assay kit. This method utilizes a modified Limulus Amebocyte Lysate (LAL) in the presence of a chromogenic substrate. In the presence of endotoxin the LAL reacts with the substrate resulting in a quantitative change in absorbance. The endotoxin level of samples was determined relative to the amount of endotoxin in a standard range using the manual of the supplier (ToxinSensor™ Chromogenic LAL Endotoxin Assay Kit, GenScript). The absorbance was measured at 540 nm (EnSpire, Perkin Elmer).

Endotoxin levels of 9 spin column purified DNA samples ranged from 3E⁻⁶ to 9E⁻³ EU/μg DNA and an average of 2.1E⁻³ EU/μg DNA. Conversely, the levels, measured for three gel-purified DNA samples, ranged from 2.6E⁻¹ to 3.5E⁻¹ EU/μg DNA, with an average of 3.2E⁻¹ EU/μg DNA. It follows that the self-immolative vector allows use of convenient techniques that also contribute to avoiding contaminants.

### 4.3 Functionality of Isolated Inserts

The functionality of backbone degraded-spin column purified fragment of interest was compared with fragment of interest generated by classical digestion-agarose purification. As example the expression of SeAP in stable mammalian cell pools was measured.

### 4.3.1 Transfection of Agarose Insert and Self-Immolation Insert

Two DNA solutions were made to compare the effect of the DNA purification method on the expression of seAP in CHO-GS−/− cells. The expression cassettes (see Example 2) were identical and harboured an intronic promoter and an additional expression regulating element (see also WO2015/102487). In one case the seAP expression cassette was isolated from the vector backbone using agarose gel isolation (see earlier Example); in the second case MluI/Bst17I digestion of the plasmid, resulting in fragmentation of the backbone, was followed by spin-column purification of the expression cassette (see method in earlier Example).

CHO-GS−/− cells (Horizon Discovery) were maintained per manufacturer's instructions. Quadruplicate transfections were performed using 3E⁶ cells in 3 ml CD CHO medium (Gibco), 5 μg of agarose-gel purified or backbone degraded/spin column purified DNA and 5 μl FreeStyle MAX Reagent (Life Technologies). Post-transfection pools were selected static in 6 well plates in 4 ml CD CHO medium (Gibco) at 37° C., 6% C02. At the start of recovery cells were transferred to T75 Flasks and cultured into 8-12 ml till a VCD of >1E⁶ cells/ml and >60% viable.

Cells were transferred to a 125 ml shake flask and cultured until cells reached a viable cell density above 90%. Stable pools were seeded in 30 ml CD forti CHO medium (Gibco) at a density of 4E⁵ cells/ml in shake flasks. The cells were cultured for 9-11 days till viable cell density dropped below 40%. The SeAP exhaust titers were determined with a SEAP Reporter Gene Assay Kit (ab13307, Abcam) using the Enspire (Perkin Elmer).

Exhaust titers of stable cell pools with backbone degraded/spin column purified DNA were 2.0 U/ml, whereas the titer of cells with the agarose fragment isolated DNA was 1.7 U/ml. So the yield of SeAP is similar irrespective whether backbone degraded/spin column purified or agarose-gel purified fragment of interest is used.

In the same experiment the effect of removing the bacterial backbone per se was also determined. Stable cell pools generated with spin column purified, linearized plasmid, i.e. without digesting the plasmid backbone, resulted in an exhaust seAP titer of 1.1 U/ml. This indicates that removal of the backbone by application of the shredder-removal method improves the performance of the expression cassette described in WO 2015/102487.

1.-18. (canceled)

19. A method for separating a polynucleotide insert from a polynucleotide vector backbone, the method comprising the steps of
i) providing a recombinant polynucleotide vector comprising the insert and the vector backbone, wherein the vector backbone comprises a first plurality of restriction sites that divide the vector backbone into fragments, wherein all fragments have a length of at most 600 bp and at least 40 bp;
ii) contacting the recombinant vector with cleavage means capable of specifically cleaving the first plurality of restriction sites to produce backbone fragments and the insert; and optionally,
iii) separating the insert from the backbone fragments of step ii).

20. The method according to claim 19, wherein the separation of step iii uses a technique selected from the group consisting of a spin column, a size exclusion column, and solid phase reversible immobilization (SPRI).

21. The method according to claim 19, wherein the cleavage means are restriction enzymes.

22. The method according to claim 21, wherein the restriction enzyme recognizes a restriction site of 6 or 7 nucleotides.

23. The method according to claim 19, wherein the cleavage means comprise 3, 2, or 1 species of restriction enzymes.

24. The method according to claim 19, wherein the insert inside does not comprise a restriction site of the first plurality of restriction sites.

25. The method according to claim 19, wherein the vector backbone further comprises a polynucleotide encoding a functional selection marker.

26. The method according to claim 24, wherein the polynucleotide encoding a functional selection marker is selected from the group consisting of SEQ ID NOs: 14-18, 79, and 110-116, or is selected from the group consisting of SEQ ID NOs: 69, 70, and 81-88.

27. The method according to claim 19, wherein the fragments have a length of at most 550 bp.

28. The method according to claim 19, wherein the fragments have a length of at most 500, 450, 400, 350, 300, 250, 200, 150, or 100 bp or less.

29. The method according to claim 19, wherein the fragments have a length of at most 550 bp and at least 60 bp.

30. The method according to claim 19, wherein the fragments have a length of at most 450 bp and at least 80 bp.

31. The method according to claim 1, wherein the fragments have a length of at most 400 bp and at least 100 bp.

32. The method according to claim 19, wherein the vector backbone further comprises a multiple cloning site that does not comprise a restriction site of the first plurality of restriction sites.

33. A polynucleotide vector backbone as defined in claim 19.

34. The polynucleotide vector backbone according to claim 33, wherein the vector backbone has at least 70% sequence identity with any one of SEQ ID NOs: 68 and 80.

35. A recombinant polynucleotide vector comprising a polynucleotide insert and the polynucleotide vector backbone as defined in claim 33.


SEQUENCE LISTING (118 sequences)

SEQ ID NO: 1 - DNA, 1835 bp, synthetic construct
  misc_feature 1..1835: note = Reference vector backbone
  source 1..1835: mol_type = other DNA
cacgcgtcttaagaccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatca

SEQ ID NO: 2 - DNA, 4470 bp, synthetic construct
  misc_feature 1..4470: note = Vector backbone
  source 1..4470: mol_type = other DNA
ctttcctgcgttatcccctgattctgtggataaccgtattaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatctcgggccccaaataatgattttattttgactgatagtgacctgttcgttgcaacaaattgatgagcaatgcttttttataatgccaagtttgtacaaaaaagcagaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtgggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 3 - DNA, 3000 bp, synthetic construct
  misc_feature 1..3000: note = Vector backbone
  source 1..3000: mol_type = other DNA
gggcgaattgggcccgacgtcgcatgctcccggccgccatggccgcgggatatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 4 - DNA, 5428 bp, synthetic construct
  misc_feature 1..5428: note = Vector backbone
  source 1..5428: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 2710 bp, synthetic construct
  misc_feature 1..2710: note = Vector backbone
  source 1..2710: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 6 - DNA, 5900 bp, synthetic construct
  misc_feature 1..5900: note = Vector backbone
  source 1..5900: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatggccagaaccagaaccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 7 - protein, 271 aa, synthetic construct
  REGION 1..271: note = Kanamycin resistance marker
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 8 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 9 - protein, 123 aa, synthetic construct
  REGION 1..123: note = Zeocin resistance marker
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQ

SEQ ID NO: 10 - protein, 219 aa, synthetic construct
  REGION 1..219: note = Chloramphenicol resistance marker
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 11 - protein, 396 aa, synthetic construct
  REGION 1..396: note = Tetracyclin resistance marker
MKSNNALIVILGTVTLDAVGIGLVMPVLPGLLRDIVHSDSIASHYGVLLALYALMQFLCAPVLGALSDRFGRRPVLLASLLGATIDYAIMATTPVLWILYAGRIVAGITGATGAVAGAYIADITDGEDRARHFGLMSACFGVGMVAGPVAGGLLGAISLHAPFLAAAVLNGLNLLLGCFLMQESHKGERRPMPLRAFNPVSSFRWARGMTIVAALMTVFFIMQLVGQVPAALWVIFGEDRFRWSATMIGLSLAVFGILHALAQAFVTGPATKRFGEKQAIIAGMAADALGYVLLAFATRGWMAFPIMILLASGGIGMPALQAMLSRQVDDDHQGQLQGSLAALTSLTSITGPLIVTAIYAASASTWNGLAWIVGAALYLVCLPALRRGAWSRATST

SEQ ID NO: 12 - protein, 263 aa, synthetic construct
  REGION 1..263: note = Streptomycin resistance marker
MREAVIAEVSTQLSEVVGVIERHLEPTLLAVHLYGSAVDGGLKPHSDIDLLVTVTVRLDETTRRALINDLLETSASPGESEILRAVEVTIVVHDDIIPWRYPAKRELQFGEWQRNDILAGIFEPATIDIDLAILLTKAREHSVALVGPAAEELFDPVPEQDLFEALNETLTLWNSPPDWAGDERNVVLTLSRIWYSAVTGKIAPKDVAADWAMERLPAQYQPVILEARQAYLGQEEDRLASRADQLEEFVHYVKGEITKVVGK

SEQ ID NO: 13 - protein, 116 aa, synthetic construct
  REGION 1..116: note = LacZ alpha marker
MTMITPSLHACRFKQSTLDLIKDPARPRVPSSNSLAVVLQRRDWENPGVTQLNRLAAHPPFASWRNSEEARTDRPSQQLRSLNGEWRLMRYFLLTHLCGISHRIWCTLSTICSDAA

SEQ ID NO: 14 - DNA, 819 bp, synthetic construct
  misc_feature 1..819: note = Kanamycin resistance marker
  source 1..819: mol_type = other DNA
atgagccatattcaaacgcgtgaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgacgcgtggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgacgcgtcagattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgcgtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 15 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacgtatacgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 16 - DNA, 375 bp, synthetic construct
  misc_feature 1..375: note = Zeocin resistance marker
  source 1..375: mol_type = other DNA
atggccaagttgaccagtgccgttccggtgctcaccgcgcgcgacgtcgccggagcggtcgagttctggaccgaccggctcgggttctcccgggacttcgtggaggacgacttcgccggtatacatcgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcgtccgggccggccatgaccgagatcggcgagcagccgtgggggcgggagttcgccctgcgcgacccggccggcaactgcgtgcacttcgtggccgaggagcaggactga

SEQ ID NO: 17 - DNA, 660 bp, synthetic construct
  misc_feature 1..660: note = Chloramphenicol resistance marker
  source 1..660: mol_type = other DNA
atggagaaaaaaatcactgggtataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcaccctttgtataccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtatacaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 18 - DNA, 351 bp, synthetic construct
  misc_feature 1..351: note = LacZ alpha marker
  source 1..351: mol_type = other DNA
atgaccatgattacgccaagcttgcatgcctgcaggtttaaacagtcgactctagacttaattaaggatccggcgcgcccacgcgtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 19 - DNA, 45 bp, synthetic construct
  misc_feature 1..45: note = MCS
  source 1..45: mol_type = other DNA
acgcgtcctgcagggcggccgcgtcgacgcgcgcacatgtgtata

SEQ ID NO: 20 - DNA, 674 bp, synthetic construct
  misc_feature 1..674: note = Ori
  source 1..674: mol_type = other DNA
atgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 21 - DNA, 675 bp, synthetic construct
  misc_feature 1..675: note = Ori
  source 1..675: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 22 - DNA, 675 bp, synthetic construct
  misc_feature 1..675: note = Ori
  source 1..675: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 23 - DNA, 676 bp, synthetic construct
  misc_feature 1..676: note = Ori
  source 1..676: mol_type = other DNA
tcagtagaaaagattaaaggatcttcttgagatcctttttttctgcgcgtaatctgctgcttgcaaacaaaaaaaccaccgctaccaacggtggtttgtttgccggatcaagagctaccaactctttttccgaaggtaactggcttcagcagagcgcagataccaaatactgtccttctagtgtagccgtagtcgggccactacttcaagaactctgtagcaccgtttgtgccatcatcgctctgctaatccggttaccagtggctgctgccagtggcgttaaggcgtgccttaccgggttggactcaagacgatagttaccggataaggcgcagcggtcgggctgaacggggggttcgtgcacacagcccagcttggagcgaacgacctacaccgaactgagataccaacagcgtgagctatgagaaagcgccacgcttcccgaagggagaaaggcggacaggtatccggtaagcggcagggtcggaacaggagagcgcacgagggagcttccagggggaaacgcctggtagctttatagtcctgtcgggtttcgccacctctgacttgagcgtctatttttgtgatgctcgtcaggggggcggagcctatggaaaaacgcctgctacgtggccttcttcctgttcctggtcttttgctcacatgttctttccgg

SEQ ID NO: 24 - DNA, 282 bp, synthetic construct
  misc_feature 1..282: note = Ori
  source 1..282: mol_type = other DNA
aagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatct

SEQ ID NO: 25 - DNA, 830 bp, synthetic construct
  misc_feature 1..830: note = Ori
  source 1..830: mol_type = other DNA
ttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgc

SEQ ID NO: 26 - DNA, 510 bp, synthetic construct
  misc_feature 1..510: note = Ori
  source 1..510: mol_type = other DNA
ggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgt

SEQ ID NO: 27 - DNA, 676 bp, synthetic construct
  misc_feature 1..676: note = Ori
  source 1..676: mol_type = other DNA
atgtgagcaaaaggccagcaaaaggccaggaaccgtaaaaaggccgcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatacaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 28 - DNA, 676 bp, synthetic construct
  misc_feature 1..676: note = Ori
  source 1..676: mol_type = other DNA
atgtgagcaaaaggccagcaaaaggccaggaaccgtaaaaaggccgcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtatacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 29 - DNA, 676 bp, synthetic construct
  misc_feature 1..676: note = Ori
  source 1..676: mol_type = other DNA
atgtgagcaaaaggccagcaaaaggccaggaaccgtaaaaaggccgcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtataccagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggg

SEQ ID NO: 30 - DNA, 173 bp, synthetic construct
  misc_feature 1..173: note = TEE
  source 1..173: mol_type = other DNA
caagctctagcaggaagaagaaataagaagaagaagaagaagaagaagaagcgtctcctcttcttcttgtgagagcaaaaaataaaactcccaaaaaaaagaaaatcatcaaaaaaacaaatttcaaaaagagtttttgtgtttggggattaaagaataaaaaaaacaacgtc

SEQ ID NO: 31 - DNA, 173 bp, synthetic construct
  misc_feature 1..173: note = TEE
  source 1..173: mol_type = other DNA
caagctctagcacgtctcctcttcttcttgtgagagtaaaaaakaaaactcccaaaaaaaakaaaatcatcaaaaaaacaaatttcaaaaagagtttttgtgtttggggattaaagaakaaaaaaaacaaggaagaagaaakaagaagaagaagaagaagaagaagaagcctc

SEQ ID NO: 32 - DNA, 274 bp, synthetic construct
  misc_feature 1..274: note = TEE
  source 1..274: mol_type = other DNA
caagctctagcaggaagaagaaakaagaagaagaagaagaagaagaagaagcgtctcctcttcttcttgtgagagtaaaaaakaaaactcccaaaaaaaakaaaatcatcaaaaaaacaaatttcaaaaagagtttttgtgtttggggattaaagaakaaaaaaaacaacaggtgagtaagcgcagttgtcgtctcttgcggtgccgttgctggttctcacaccttttaggtctgttctcgtcttccgttctgactctctctttttcgttgcag

SEQ ID NO: 33 - DNA, 277 bp, synthetic construct
  misc_feature 1..277: note = TEE
  source 1..277: mol_type = other DNA
caagctctagcaggaagaagaaakaagaagaagaagaagaagaagaagaagcgtctcctcttcttcttgtgagagtaaaaaakaaaactcccaaaaaaaagaaaatcatcaaaaaaacaaatttcaaaaagagtttttgtgtttggggattaaagaagaaaaaaaacaacaggtgagtaagcgcagttgtcgtctcttgcggtgccgttgctggttctcacaccttttaggtctgttctcgtcttccgttctgactctctctttttcgttgcaggcc

SEQ ID NO: 34 - DNA, 278 bp, synthetic construct
  misc_feature 1..278: note = TEE
  source 1..278: mol_type = other DNA
caagctctagcaggaagaagaaagaagaagaagaagaagaagaagaagaagcgtctcctcttcttcttgtgagagtaaaaaagaaaactcccaaaaaaaagaaaatcatcaaaaaaacaaatttcaaaaagagtttttgtgtttggggattaaaggtgagtaagcgcagttgtcgtctcttgcggtgccgttgctggttctcacaccttttaggtctgttctcgtcttccgttctgactctctctttttcgttgcagaactcctaaaaaaccgccacc

SEQ ID NO: 35 - DNA, 266 bp, synthetic construct
  misc_feature 1..266: note = TEE
  source 1..266: mol_type = other DNA
caagctctagcaggaagaagaaakaagaagaagaagaagaagaagaagaagcgtctcctcttcttcttgtgagagtaaaaaakaaaactcccaaaaaaaagaaaatcatcaaaaaaacaaatttcaaaaagagtgaggtaagattatcgatatttaaattatttatttcttcttttccatttttttggctaacattttccatggttttatgatatcatgcaggtacgttttgtgtttggggattaaagaataaaaaaaacaaaaca

SEQ ID NO: 36 - DNA, 178 bp, synthetic construct
  misc_feature 1..178: note = TEE
  source 1..178: mol_type = other DNA
cgtctcctcttcttcttgtgagagtaaaaaakaaaactcccaaaaaaaagaaaatcatcaaaaaaacaaatttcaaaaagagtttttgtgtttggggattaaagaakaaaaaaaacaacctcgtgcgtgttgccgattcgcgtacgaatacgccttgtgctgacacttctgtagcacc

SEQ ID NO: 37 - DNA, 86 bp, synthetic construct
  misc_feature 1..86: note = TEE
  source 1..86: mol_type = other DNA
ttcaaaaagagtttttgtgtttggggattaaagaataaaaaaaacaaggaagaagaaakaagaagaagaagaagaagaagaagaag

SEQ ID NO: 38 - DNA, 277 bp, synthetic construct
  misc_feature 1..277: note = TEE
  source 1..277: mol_type = other DNA
caagctctagcacgtctcctcttcttcttgtgagagtaaaaaakaaaactcccaaaaaaaagaaaatcatcaaaaaaacaaatttcaaaaagagtttttgtgtttggggattaaagaagaaaaaaaacaaggaagaagaaakaagaagaagaagaagaagaagaagaagcaggtgagtaagcgcagttgtcgtctcttgcggtgccgttgctggttctcacaccttttaggtctgttctcgtcttccgttctgactctctctttttcgttgcaggcc

SEQ ID NO: 39 - DNA, 265 bp, synthetic construct
  misc_feature 1..265: note = TEE
  source 1..265: mol_type = other DNA
caagctctagcacgtctcctcttcttcttgtgagagtaaaaaakaaaactcccaaaaaaaagaaaatcatcaaaaaaacaaatttcaaaaagagtgaggtaagattatcgatatttaaattatttatttcttcttttccatttttttggctaacattttcctaggttttattatatctagcaggtacgttttgtgtttggggattaaagaagaaaaaaaacaaggaagaagaaakaagaagaagaagaagaagaagaagaaaaca

SEQ ID NO: 40 - DNA, 188 bp, synthetic construct
  misc_feature 1..188: note = TEE
  source 1..188: mol_type = other DNA
caasctctaccaccaasaacaaacaacaacaacatatayaaaacaacaacmaccatctcctcttcttcttgtcaastmmaaaaycaaactcccaaaaaaaagmaaatcatcaaaammacaaatttcaaamaacaacawyaaacaacaaamaamattaacatcatatcaaggcggccgcccccttcacc

SEQ ID NO: 41 - DNA, 173 bp, synthetic construct
  misc_feature 1..173: note = TEE
  source 1..173: mol_type = other DNA
caagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacgtc

SEQ ID NO: 42 - DNA, 175 bp, synthetic construct
  misc_feature 1..175: note = TEE
  source 1..175: mol_type = other DNA
caagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacgtccc

SEQ ID NO: 43 - DNA, 189 bp, synthetic construct
  misc_feature 1..189: note = TEE
  source 1..189: mol_type = other DNA
agatcactagaagcttcaagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacgcc

SEQ ID NO: 44 - DNA, 189 bp, synthetic construct
  misc_feature 1..189: note = TEE
  source 1..189: mol_type = other DNA
agatcactagaagcttcaagctctagcaggaagaagaaagaagaagaagaagaagaagaagaagaagcgtctcctcttcttcttgtgagagtaaaaaagaaaactcccaaaaaaaagaaaatcatcaaaaaaacaaatttcaaaaagagtttttgtgtttggggattaaagaagaaaaaaaacaacgcc

SEQ ID NO: 45 - DNA, 191 bp, synthetic construct
  misc_feature 1..191: note = TEE
  source 1..191: mol_type = other DNA
agatcactagaagcttcaagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacaggcc

SEQ ID NO: 46 - DNA, 284 bp, synthetic construct
  misc_feature 1..284: note = TEE
  source 1..284: mol_type = other DNA
ctttttcgcaacgggtttgccgccagaacacaggtgtcgtgaggaattagcttggtactaatacgactcactatagggagacccaagctggctaggtaagcttggtacccaagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacgtccc

SEQ ID NO: 47 - DNA, 230 bp, synthetic construct
  misc_feature 1..230: note = TEE
  source 1..230: mol_type = other DNA
aacccactgcttactggcttatcgaaattaatacgactcactatagggagacccaagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacctccacc

SEQ ID NO: 48 - DNA, 177 bp, synthetic construct
  misc_feature 1..177: note = TEE
  source 1..177: mol_type = other DNA
caagctctagcagcaacaacaaataacaacaacaacaacaacaacaacaagcgtctcctcttcttcttgtgagagtaaaaaataaaactcccaaaaaaaagaaaatcatcaaaaaaacaaatttcaaaaagagtttttgtgtttggggattaaagaataaaaaaaacaacctccacc

SEQ ID NO: 49 - DNA, 191 bp, synthetic construct
  misc_feature 1..191: note = TEE
  source 1..191: mol_type = other DNA
agatcactagaagcttcaagctctagcaggaagaagaaataagaagaagaagaagaataagaagaagcgtctcgtcttcttcttgtgagagtaaaaaataaaactcccaaaaaaaataaaatcatcaaaaaaagaaatttcaaaaagagtttttgtgtttggggattaaagaataaaaaaaacaacaggcc

SEQ ID NO: 50 - DNA, 189 bp, synthetic construct
  misc_feature 1..189: note = TEE
  source 1..189: mol_type = other DNA
agatcactagaagcttcaagctctagcaggaagaagaaataataagaagaagaagaataagaagaagcgtctcctcttcttcttgtgagagtaaaaaataaaactcccaaaaaaaataaaatcatcaaaaaaataaatttcaaaaagagtttttgtgtttggggattaaagaataaaaaaaacaacgcc

SEQ ID NO: 51 - DNA, 173 bp, synthetic construct
  misc_feature 1..173: note = TEE
  source 1..173: mol_type = other DNA
caagctctagcaggaagaagaaakaagaagaagaagaagaagaagaagaagcgtctcctcttcttcttgtgagagtaaaaaakaaaactcccaaaaaaaakaaaatcatcaaaaaaacaaatttcaaaaagagtttttgtgtttggggattaaagaakaaaaaaaacaacgtc

SEQ ID NO: 52 - DNA, 274 bp, synthetic construct; identical to SEQ ID NO: 32
  misc_feature 1..274: note = TEE
  source 1..274: mol_type = other DNA

SEQ ID NO: 53 - DNA, 123 bp, synthetic construct
  misc_feature 1..123: note = TEE
  source 1..123: mol_type = other DNA
ggcgtctcctcttcttcttgtgagagtaaaaaataaaactcccaaaaaaaakaaaatcatcaaaaaaacaaatttcaaaaagagtttttgtgtttggggattaaagaakaaaaaaacaacgtc

SEQ ID NO: 54 - DNA, 384 bp, synthetic construct
  misc_feature 1..384: note = TEE
  source 1..384: mol_type = other DNA
caagctctagcaggaagaagaaataagaagaagaagaagaagaagaagaagcgtctcctcttcttcttgtgagagtaaaaaataaaactcccaaaaaaaagaaaatcatcaaaaaaacaaatttcaaaaagagtttttgtgtttggggattaaagaataaaaaaaacaacgtctggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccg

SEQ ID NO: 55 - DNA, 2522 bp, synthetic construct
  misc_feature 1..2522: note = EEE1+CMV+TEE
  source 1..2522: mol_type = other DNA
tttcaggcaaccagagctacatagtgagatcctgtctcaacaaaaataaaataatctaaggcttcaaagggttcaatctcttaggtagctaaatatgaacaaaatttgggaaatgtgaccttttccttagtgacagtcagatagaaccttctcgagtgcaaggacaccaagtgcaaacaggctcaagaacagcctggaaaggtctagtgctatggggcttcaggtcgaatgccaactgttttcaagaactgtgtggatttttctgcctgtaacgaattcagattcatttttcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggtaagcgggtgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccctgtttaaacttaaacaagtttgtacaaaaaagcaggctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatagtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagcttcaagctctagcaggaagaagaaagaagaagaagaagaagaagaagaagaagcgtctcctcttcttcttgtgagagtaaaaaagaaaactcccaaaaaaaagaaaatcatcaaaaaaacaaatttcaaaaagagtttttgtgtttggggattaaagaagaaaaaaaacaacaggtgagtaagcgcagttgtcgtctcttgcggtgccgttgctggttctcacaccttttaggtctgttctcgtcttccgttctgactctctctttttcgttgcaggcc

SEQ ID NO: 56 - DNA, 3321 bp, synthetic construct
  misc_feature 1..3321: note = EEE1-Xt+CMV+TEE
  source 1..3321: mol_type = other DNA
tggtgaccctgtctcaaaaaaccctcaaaaagtgttgggattagtggcatgcaccaccattcccaccaaaggtttatttttaataatatgtgtgtgagtgtgtatcactatgagtatatgtcaatatgtgtcaatgtccccagggacatttaaagagcccctgaagctggagtcataggccattatgaactgcctgacatggctaatgggaattgaactcagattttctggaagttatacctgctcttactgctgagccatgtctctgaagaccccagggatttttttttttttttgagacaggtattttctgtatagccctggctgtcctgaaagcactctctatatgtagaccaggcttgcctggagcttggatatgcacctgcttctgcctcaggaatggtgggattgaaggtgtgcaccaccacatccgctaacatgcacaattcttaatgggtttatatcttatttaatgaatgaaaggtttgggggatggatgtagcttaatggaaaatgactgaagatttcaattaaaaatctggggcttagctgcgcggtgggtggtgcctgcctttagtcccagtactggggaggcagaggaaggaggatctctgtgagttcgaggccagctggtctataacgtgagttccaggacagccagagatacacagacaaaccctgtctcaccaaaacaaaacaacaacaacaacaacaaatctgggacgtaggcttggtgtggtggcacacattttgattccagcacttggaaggaagaggcctgcatggtctacatagcttgtttcaggcaaccagagctacatagtgagatcctgtctcaacaaaaataaaataatctaaggcttcaaagggttcaatctcttaggtagctaaatatgaacaaaatttgggaaatgtgaccttttccttagtgacagtcagatagaaccttctcgagtgcaaggacaccaagtgcaaacaggctcaagaacagcctggaaaggtctagtgctatggggcttcaggtcgaatgccaactgttttcaagaactgtgtggatttttctgcctgtaacgaattcagattcatttttcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggtaagcgggtgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccctgtttaaacttaaacaagtttgtacaaaaaagcaggctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatagtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagcttcaagctctagcaggaagaagaaagaagaagaagaagaagaagaagaagaagcgtctcctcttcttcttgtgagagtaaaaaagaaaactcccaaaaaaaagaaaatcatcaaaaaaacaaatttcaaaaagagtttttgtgtttggggattaaagaagaaaaaaaacaacaggtgagtaagcgcagttgtcgtctcttgcggtgccgttgctggttctcacaccttttaggtctgttctcgtcttccgttctgactctctctttttcgttgcaggcc

SEQ ID NO: 57 - DNA, 2232 bp, synthetic construct
  misc_feature 1..2232: note = EEE1-80+CMV+TEE
  source 1..2232: mol_type = other DNA
tcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggtaagcgggtgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccctgtttaaacttaaacaagtttgtacaaaaaagcaggctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatagtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagcttcaagctctagcaggaagaagaaagaagaagaagaagaagaagaagaagaagcgtctcctcttcttcttgtgagagtaaaaaagaaaactcccaaaaaaaagaaaatcatcaaaaaaacaaatttcaaaaagagtttttgtgtttggggattaaagaagaaaaaaaacaacaggtgagtaagcgcagttgtcgtctcttgcggtgccgttgctggttctcacaccttttaggtctgttctcgtcttccgttctgactctctctttttcgttgcaggcc

SEQ ID NO: 58 - DNA, 1942 bp, synthetic construct
  misc_feature 1..1942: note = EEE1-60+CMV+TEE
  source 1..1942: mol_type = other DNA
cgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccctgtttaaacttaaacaagtttgtacaaaaaagcaggctagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatagtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagcttcaagctctagcaggaagaagaaagaagaagaagaagaagaagaagaagaagcgtctcctcttcttcttgtgagagtaaaaaagaaaactcccaaaaaaaagaaaatcatcaaaaaaacaaatttcaaaaagagtttttgtgtttggggattaaagaagaaaaaaaacaacaggtgagtaagcgcagttgtcgtctcttgcggtgccgttgctggttctcacaccttttaggtctgttctcgtcttccgttctgactctctctttttcgttgcaggcc

SEQ ID NO: 59 - DNA, 1449 bp, synthetic construct
  misc_feature 1..1449: note = EEE1
  source 1..1449: mol_type = other DNA
tttcaggcaaccagagctacatagtgagatcctgtctcaacaaaaataaaataatctaaggcttcaaagggttcaatctcttaggtagctaaatatgaacaaaatttgggaaatgtgaccttttccttagtgacagtcagatagaaccttctcgagtgcaaggacaccaagtgcaaacaggctcaagaacagcctggaaaggtctagtgctatggggcttcaggtcgaatgccaactgttttcaagaactgtgtggatttttctgcctgtaacgaattcagattcatttttcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggtaagcgggtgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccct

SEQ ID NO: 60 - DNA, 1228 bp, synthetic construct
  misc_feature 1..1228: note = EEE2
  source 1..1228: mol_type = other DNA
gtaaagcagatcacacagaatatggcacacttgagcacttgatgtgtactacattactcttagtgacgactttaattatcgtgcgcattcccagcgcttcctatggtgcccaacacagagcggacgcctagagacaattttgggggatggggcagatgctctgcctcgggaaaaaaaaagcacacctgccctgacgttggtggctgggtctggaagatacgtggaaattaagctaaggatgtgtggcttccagatcaaaaaccgcaaaaatctaacgccgtgactactgactacggtcagagagcacagactggagcaacctctcacggcctgggctgtctgcgcgtgcgtgagccagaaacccgaggggctccctgggcccgccctatcgatcgacccgatcggggatcgtcagcttggttctggccacagaggttgctcttctcgcgatgcttcagacctggcggcagggaaagggtgggctaattggagagccaggaagagcgtgaggcggccccacgctgctttcccagaaggctgtgcgtgctcctcgcttcctccgcggtcttccgagcggtcgcgtgaactgcttccagcaggctggccatggcgcttcacgttcccaaggctccgggctttgcccagatgctcaaggagggagcgaaagtaagggctgaaggaaaggaatgaggtgggagcgtcagcatagggctgcggcggcggcggcgaagtaggagggcctactaacgggctgagcgtgctgccctggctcagcggccgggggaagagaagattccagaaagggaggtgattttggaagggctcggccaccggagcctgcgggcacttctcttcttccgcgaccgggagaaggccgagggatcggcggcacgatcgacattgtacaccttgaaggtggacggatgtgaagccgcgcgtgcgttttgcctccatccgtaaatggggctaaggcccgtcacccttaaaggaggttgtgagggtgaaattgaataacgtagatgaaattgtcttgagaactgcgacgtcgattatcacatagctcgcgagttgtaggatggggaagaacgagaactagccgatccagagaagagagtgggaaaaagggccgggtcttggttgcttgcttcccagtgagaaacatacggctttcagcttagttgacagaagccatgcgttgtagccaaatgagttccggtcccaacttatg

SEQ ID NO: 61 - DNA, 753 bp, synthetic construct
  misc_feature 1..753: note = CMV promoter
  source 1..753: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatagtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctt

SEQ ID NO: 62 - DNA, 69 bp, synthetic construct
  misc_feature 1..69: note = minimal CMV promoter
  source 1..69: mol_type = other DNA
taggcgtgtacggtgggaggtctatataagcagagctcgtttagtgaaccgtcagatcactagaagctt

SEQ ID NO: 63 - DNA, 2248 bp, synthetic construct
  misc_feature 1..2248: note = EEE1-XT
  source 1..2248: mol_type = other DNA
tggtgaccctgtctcaaaaaaccctcaaaaagtgttgggattagtggcatgcaccaccattcccaccaaaggtttatttttaataatatgtgtgtgagtgtgtatcactatgagtatatgtcaatatgtgtcaatgtccccagggacatttaaagagcccctgaagctggagtcataggccattatgaactgcctgacatggctaatgggaattgaactcagattttctggaagttatacctgctcttactgctgagccatgtctctgaagaccccagggatttttttttttttttgagacaggtattttctgtatagccctggctgtcctgaaagcactctctatatgtagaccaggcttgcctggagcttggatatgcacctgcttctgcctcaggaatggtgggattgaaggtgtgcaccaccacatccgctaacatgcacaattcttaatgggtttatatcttatttaatgaatgaaaggtttgggggatggatgtagcttaatggaaaatgactgaagatttcaattaaaaatctggggcttagctgcgcggtgggtggtgcctgcctttagtcccagtactggggaggcagaggaaggaggatctctgtgagttcgaggccagctggtctataacgtgagttccaggacagccagagatacacagacaaaccctgtctcaccaaaacaaaacaacaacaacaacaacaaatctgggacgtaggcttggtgtggtggcacacattttgattccagcacttggaaggaagaggcctgcatggtctacatagcttgtttcaggcaaccagagctacatagtgagatcctgtctcaacaaaaataaaataatctaaggcttcaaagggttcaatctcttaggtagctaaatatgaacaaaatttgggaaatgtgaccttttccttagtgacagtcagatagaaccttctcgagtgcaaggacaccaagtgcaaacaggctcaagaacagcctggaaaggtctagtgctatggggcttcaggtcgaatgccaactgttttcaagaactgtgtggatttttctgcctgtaacgaattcagattcatttttcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggtaagcgggtgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccct

SEQ ID NO: 64 - DNA, 724 bp, synthetic construct
  misc_feature 1..724: note = EEE1-50
  source 1..724: mol_type = other DNA
tctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccct

SEQ ID NO: 65 - DNA, 1449 bp, synthetic construct
  misc_feature 1..1449: note = EEE1-SL
  source 1..1449: mol_type = other DNA
tttcaggcaaccagagctacatagtgagatcctgtctcaacaaaaataaaataatctaaggcttcaaagggttcaatctcttaggtagctaaatatgaacaaaatttgggaaatgtgaccttttccttagtgacagtcagatagaaccttctcgagtgcaaggacaccaagtgcaaacaggctcaagaacagcctggaaaggtctagtgctatggggcttcaggtcgaatgccaactgttttcaagaactgtgtggatttttctgcctgtaacgaattcagattcatttttcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggaaagcggttgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctgctgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaattgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccct

SEQ ID NO: 66 - DNA, 455 bp, synthetic construct
  misc_feature 1..455: note = EF1a promoter
  source 1..455: mol_type = other DNA
ggatccttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 67 - DNA, 2617 bp, synthetic construct
  misc_feature 1..2617: note = Transcription regulating sequence
  source 1..2617: mol_type = other DNA
tttcaggcaaccagagctacatagtgagatcctgtctcaacaaaaataaaataatctaaggcttcaaagggttcaatctcttaggtagctaaatatgaacaaaatttgggaaatgtgaccttttccttagtgacagtcagatagaaccttctcgagtgcaaggacaccaagtgcaaacaggctcaagaacagcctggaaaggtctagtgctatggggcttcaggtcgaatgccaactgttttcaagaactgtgtggatttttctgcctgtaacgaattcagattcatttttcaaaactcggggagagttttccccctttataattttttttttaaatttattaaactttgtttcgttccccttgttttgagaattgcagagtcatccaccctgtcacagtgccagggagctcagggatgggcccaggggcctggcggggctgaaggggctggggaagcgagggctccaaagggaccccagtgtggcaggagccaaagccctaggtccctagaacgcagaggccaccgggaccccccagacggggtaagcgggtgggtgtctggggcgcgaagccgcactgcgcatgcgccgaggtccgctccggccgcgctgatccaagccgggttctcgcgccgacctggtcgtgattgacaagtcacacacgctgatccctccgcggggccgcacagggtcacagcctttcccctccccacaaagccccctactctctgggcaccacacacgaacattccttgagcgtgaccttgttggctctagtcaggcgcctccggtgcagagactggaacggccttgggaagtagtccctaaccgcatttccgcggagggatcgtcgggagggcgtggcttctgaggattatataaggcgactccgggcgggtcttagctagttccgtcggagacccgagttcagtcgccgcttctctgtgaggactgctgccgccgccgctggtgaggagaagccgccgcgcttggcgtagctgagagacggggagggggcgcggacacgaggggcagcccgcggcctggacgttctgtttccgtggcccgcgaggaaggcgactgtcctgaggcggaggacccagcggcaagatggcggccaagtggaagcctgaggggataggcgagcggccctgaggcgctcgacggggttgggggggaagcaggcccgcgaggcagctgcagccgggaacgtgcggccaaccccttattttttttgacgggttgcgggccgtaggtgcctccgaagtgagagccgtgggcgtttgactgtcgggagaggtcggtcggattttcatccgttgctaaagacggaagtgcgactgagacgggaaggggggggagtcggttggtggcggttgaacctggactaaggcgcacatgacgtcgcggtttctatgggctcataatgggtggtgaggacatttccctgactatagctttccctcagttgtaggacagggtttgggcctcggcctcgggttaggctctccagagtgggcaggaaccggaaatccagaggggggaaaagtgagcctaaattgagttttgtttcttgtcctatatggtttagagagagactcgctgcaaaaccgtggctggcctggaactctagaccagaaccctggcctttgccgacccacatgattagattcaaggcctgtgccaccagcccaggctttattattatggtctgggatttctgcgatttcatccctggtgttttgggatgatgacttgtgggtcttccctcctcccccttactgtttctgtccatggcgtgtgttctaacccaagtttgttcttttgggggggtgggagggttgcgataaaatgggatctatctctgccctcccaacttgagatctgcctgtcagaagtctcagtgctgagaataaaggtgtgcattggctcagacctcgattttttttttttttattattttgtaggaagtctgtagtccttacttgatacataagaccagacaggatctgatttcctgcctatgaatggtagatcctctcagtgactgcagtgtgaatggggaccacgcttttctccaaactatgcagatagccatgaaagccatgaaatgactttcagccactggtactgcaatatccactcaccatttattatatggaccaggttcaccatgcctaggtggctttgcttttgagacacggtttctctgtgtagccttggttatgtttttttgtttgtttttttaattatttttggtttttcgagacagggtttctctgtgtagctttggagcctatcctggcacttgctccggagaccaggctggcctccaactcagatctgcctgcctctgcctcccgactgctgggattaaagtaaagccattctgcaaccctgaataccactcaataggtttcttatttgaaatgtggttttatgatttttatttctggatttagaaaagaaatcttcagacagaagtcttcagacagaaactagctgtagtttggctgtgtgaactaaattggcatccatttcacagcaatccaactgttagtaccataccacgaatatttgtcattcctgacctgttttttgtttgtgtgtgtgacag

SEQ ID NO: 68 - DNA, 1886 bp, synthetic construct
  misc_feature 1..1886: note = Backbone vector
  source 1..1886: mol_type = other DNA
cacgcgtcttaagaccatgtgagcaaaaggccagcaaaaggccaggaaccgtaaaaaggccgcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatacaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcgtcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgacgcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatctgacgcgtcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccacgcgtcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcacgcgtttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcaacgcgtcctgcagggcggccgcgtcgacgcgcgcacatgtgtata

SEQ ID NO: 69 - protein, 272 aa, synthetic construct
  REGION 1..272: note = Kanamycin resistance marker
MSHIQTRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 70 - protein, 272 aa, synthetic construct
  REGION 1..272: note = Kanamycin resistance marker
MSHIQRETRSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 71 - DNA, 441 bp, synthetic construct
  misc_feature 1..441: note = Gene block 1a
  source 1..441: mol_type = other DNA
gcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 72 - DNA, 466 bp, synthetic construct
  misc_feature 1..466: note = Gene block 2a
  source 1..466: mol_type = other DNA
agttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcacc

SEQ ID NO: 73 - DNA, 488 bp, synthetic construct
  misc_feature 1..488: note = Gene block 3a
  source 1..488: mol_type = other DNA
ttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggt

SEQ ID NO: 74 - DNA, 451 bp, synthetic construct
  misc_feature 1..451: note = Gene block 4a
  source 1..451: mol_type = other DNA
ggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggc

SEQ ID NO: 75 - DNA, 164 bp, synthetic construct
  misc_feature 1..164: note = Gene block 5a
  source 1..164: mol_type = other DNA
catgacattaacctataaaaataggcgtatcaacgcgtcctgcagggcggccgcgtcgacgcgcgcacatgtgtatacacgcgtcttaagaccatgtgagcaaaaggccagcaaaaggccaggaaccgtaaaaaggccgcgttgctggcgtttttccataggct

SEQ ID NO: 76 - DNA, 488 bp, synthetic construct
  misc_feature 1..488: note = Gene block 3b
  source 1..488: mol_type = other DNA
ttctgtaatgaaggagaaaactcaccgaggcagttccataggatggcaagatcctggtatcggtctgcgattccgacgcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatctgacgcgtcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccacgcgtcatcaggagtacggataaaatgcttgatggt

SEQ ID NO: 77 - DNA, 454 bp, synthetic construct
  misc_feature 1..454: note = Gene block 4b
  source 1..454: mol_type = other DNA
ggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcacgcgtttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggc

SEQ ID NO: 78 - DNA, 454 bp, synthetic construct
  misc_feature 1..454: note = Gene block 4c
  source 1..454: mol_type = other DNA
ggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagaacgcgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggc

SEQ ID NO: 79 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 80 - DNA, 1895 bp, synthetic construct
  misc_feature 1..1895: note = Backbone vector
  source 1..1895: mol_type = other DNA
gacgcgtcttaagaccatgtgagcaaaaggccagcaaaaggccaggaaccgtaaaaaggccgcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatacaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcgtcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgtatacgtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaagacgcgttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccacgcgtgctcccaactgatcttcagcatcttttactttcaccagcgtttctgggtgagcaaaaacaggaaggcaaaatgccgcaaaaaagggaataagggcgacacggaaatgctggatgctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcaacgcgtcctgca

SEQ ID NO: 81 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGTRGSRGIIAALGPDGKPSRIVVIYTTGSQATMDERNRQIAEIGASLIKHW

SEQ ID NO: 82 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGTRGIIAALGPDGKPSRIVVIYTTGSQATMDERNRQIAEIGASLIKHW

SEQ ID NO: 83 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPTRIVVIYTTGSQATMDERNRQIAEIGASLIKHW

SEQ ID NO: 84 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTRSQATMDERNRQIAEIGASLIKHW

SEQ ID NO: 85 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDTRNRQIAEIGASLIKHW

SEQ ID NO: 86 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERTRQIAEIGASLIKHW

SEQ ID NO: 87 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPRIRSALPAGWFIADKSGAGERGSRGIIAALGPDGKPSRIVVIYTTGSQATMDERNRQIAEIGASLIKHW

SEQ ID NO: 88 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERERVIAEIGASLIKHW

SEQ ID NO: 89 - protein, 286 aa, synthetic construct
  REGION 1..286: note = Ampicillin resistance marker
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSDASMDERNRQIAEIGASLIKHW

SEQ ID NO: 90 - DNA, 441 bp, synthetic construct
  misc_feature 1..441: note = Gene block 1d
  source 1..441: mol_type = other DNA
gcgttgctggcgtttttccataggcttcgcgcccctgacgagcatcacaaaaatcgacgctcaagtcagaggacgcgtaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 91 - DNA, 443 bp, synthetic construct
  misc_feature 1..443: note = Gene block 1e
  source 1..443: mol_type = other DNA
gcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 92 - DNA, 443 bp, synthetic construct
  misc_feature 1..443: note = Gene block 1b
  source 1..443: mol_type = other DNA
gcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctacgcgtgtgtgcacgaaccccccgttcacgcgtgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 93 - DNA, 441 bp, synthetic construct
  misc_feature 1..441: note = Gene block 1c
  source 1..441: mol_type = other DNA
gcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgacccgacgcgttaccggatacctgtccgcctttctcccttcgggaagcgtggcgctttctcatagctcacgctgtaggtatctcagttgcgagtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 94 - DNA, 444 bp, synthetic construct
  misc_feature 1..444: note = Gene block 1f
  source 1..444: mol_type = other DNA
gcgttgctggcgtttttccataggctccgcccccctgacgagcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctacgcgtgtgtgcacgaaccccccgttcacgcgtgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 95 - DNA, 442 bp, synthetic construct
  misc_feature 1..442: note = Gene block 1g
  source 1..442: mol_type = other DNA
gcgttgctggcgtttttccataggctccgcccccctgacgagcatcacaaaaatcgacgcgtcaagtcagaggtggcgaaacccgacaggactataaagataccaggcgtttccccctggaagctccctcgtgcgctctcctgttccgacccgacgcgttaccggatacctgtccgcctttctcccttcgggaagcgtggcgctttctcatagctcacgctgtaggtatctcagttgcgagtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 96 - DNA, 467 bp, synthetic construct
  misc_feature 1..467: note = Gene block 2b
  source 1..467: mol_type = other DNA
agttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgcgtcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcacc

SEQ ID NO: 97 - DNA, 443 bp, synthetic construct
  misc_feature 1..443: note = Gene block 1n
  source 1..443: mol_type = other DNA
gcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtataccagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 98 - DNA, 443 bp, synthetic construct
  misc_feature 1..443: note = Gene block 1o
  source 1..443: mol_type = other DNA
gcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtatacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactac

SEQ ID NO: 99 - DNA, 443 bp, synthetic construct
  misc_feature 1..443: note = Gene block 1p
  source 1..443: mol_type = other DNA
gcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatacaggcggtgctacagagttcttgaagtggtggcctaactac

SEQ ID NO: 100 - DNA, 1557 bp, synthetic construct
  misc_feature 1..1557: note = seAP coding sequence
  source 1..1557: mol_type = other DNA
atgttgctgctcctgcttcttcttgggcttaggcttcaacttagtcttgggataattccggttgaagaagagaaccctgatttttggaatagagaggccgccgaagccttgggtgccgctaagaagctgcaacctgcacaaactgctgcgaaaaaccttatcatcttccttggtgatggaatgggagtgtctacagtgaccgctgcgcgcatcctcaagggacagaagaaggacaaattggggccagagatcccgctggcaatggacagatttccttatgtggccctgagcaagacttacaatgtcgacaagcacgtccccgattcaggggctacagctacagcatatctgtgcggtgtaaagggcaatttccagaccatcggcctcagcgccgctgcaagattcaatcaatgcaacaccactcgagggaacgaagtgatctcagtgatgaacagagccaaaaaggccggaaagtctgttggtgtcgtgacgactacccgcgtgcagcatgctagccctgctggtacatacgctcatacagtcaaccggaactggtacagcgacgccgacgttcccgcgtcagcacgccaagagggttgccaggatattgcaactcaacttatcagcaacatggatattgatgtgattttgggaggaggacgcaagtatatgtttaggatgggcaccccagatccagaatatccagacgattactcacaagggggcactcggctggacggcaagaacctggttcaggaatggcttgccaaacgacagggagcgaggtatgtctggaataggactgagcttatgcaagcatccctcgacccctccgtaacccatctgatgggactctttgagcctggggatatgaagtatgaaattcaccgcgactccacattggacccatcactgatggagatgacagaggccgcacttcgcctcctgtcacggaaccctcgaggcttctttctgttcgttgaaggagggaggatagaccacggacaccacgaaagtagggcctatagagcactcacggaaaccattatgttcgacgacgctatcgaaagggctggtcagcttacctctgaggaggacaccctctccctggtgacagccgaccattcccacgtgttcagcttcggcggctacccacttagggggtcctctatttttggactggcgccaggcaaagcccgggataggaaggcttacaccgtgttgctctacgggaacggtcccggctacgtcctgaaggacggtgcgaggcccgacgtcacagagagcgagtctggaagccctgagtataggcaacaatcagcagtacccctcgacgaggagactcacgcaggcgaggatgtcgctgtgttcgccagagggccacaggcccacctggtccacggagtgcaagagcaaactttcatcgctcacgtcatggcattcgccgcttgccttgagccctacacggcatgtgacctggcccctccggcagggacaactgacgctgctcacccaggttacagtcgcgtgggcgctgctggcaggttcgagcagacc

SEQ ID NO: 101 - DNA, 1968 bp, synthetic construct
  misc_feature 1..1968: note = GS selection marker
  source 1..1968: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggcaacaggtgagtaagcgcagttgtcgtctcttgcggtgccgttgctggttctcacaccttttaggtctgttctcgtcttccgttctgactctctctttttcgttgcaggccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgatatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtacactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgcagaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaagctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 102 - protein, 10 aa, synthetic construct
  REGION 1..10: note = N-terminal amino acids kanamycin resistance marker
MSHIQRETSC

SEQ ID NO: 103 - protein, 11 aa, synthetic construct
  REGION 1..11: note = N-terminal amino acidskanamycin resistance marker variant
MSHIQTRETSC

SEQ ID NO: 104 - protein, 11 aa, synthetic construct
  REGION 1..11: note = N-terminal amino acidskanamycin resistance marker variant
MSHIQRETRSC

SEQ ID NO: 105 - DNA, 816 bp, synthetic construct
  misc_feature 1..816: note = Kanamycin resistance marker wt CDS
  source 1..816: mol_type = other DNA
atgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 106 - DNA, 858 bp, synthetic construct
  misc_feature 1..858: note = Ampicillin resistance marker wt CDS
  source 1..858: mol_type = other DNA
atgagtattcaacatttccgtgtcgcccttattcccttttttgcggcattttgccttcctgtttttgctcacccagaaacgctggtgaaagtaaaagatgctgaagatcagttgggagcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattgg

SEQ ID NO: 107 - DNA, 375 bp, synthetic construct
  misc_feature 1..375: note = Zeocin resistance marker wt CDS
  source 1..375: mol_type = other DNA
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 108 - DNA, 660 bp, synthetic construct
  misc_feature 1..660: note = Chloramphenicol resistance marker wt CDS
  source 1..660: mol_type = other DNA
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaa

SEQ ID NO: 109 - DNA, 351 bp, synthetic construct
  misc_feature 1..351: note = LacZalpha marker
  source 1..351: mol_type = other DNA
atgaccatgattacgccaagcttgcatgcctgcaggtttaaacagtcgactctagacttaattaaggatccggcgcgcccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 110 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtacgcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 111 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggacgcgtggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 112 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagcccacgcgtatcgtagttatctacacgacggggagtcaggcaactatggatgaacgaaatagacagatcgctgagataggtgcctcactgattaagcattggtaa

SEQ ID NO: 113 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgcgtagtcaggcaactatggatgaacgaaatagacagatcgctgagataggtgcctcactgattaagcattggtaa

SEQ ID NO: 114 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatacgcgtaatagacagatcgctgagataggtgcctcactgattaagcattggtaa

SEQ ID NO: 115 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaacgcgtcagatcgctgagataggtgcctcactgattaagcattggtaa

SEQ ID NO: 116 - DNA, 861 bp, synthetic construct
  misc_feature 1..861: note = Ampicillin resistance marker
  source 1..861: mol_type = other DNA
atgagcatccagcatttccgtgtcgcccttattcccttttttgcggcattttgccttcctgtttttgctcacccagaaacgctggtgaaagtaaaagatgctgaagatcagttgggagcacgcgtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaacgcgtcttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgagaacgcgtgatcgctgagataggtgcctcactgattaagcattggtaa

SEQ ID NO: 117 - DNA, 443 bp, synthetic construct
  misc_feature 1..443: note = Gene block 1i
  source 1..443: mol_type = other DNA
gcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgcgactggcagcagccacgcgtaacaggattagcagagcgaggtatgtaggcggtgctacagagttcttgaagtggtggcctaactac

SEQ ID NO: 118 - DNA, 444 bp, synthetic construct
  misc_feature 1..444: note = Gene block 1l
  source 1..444: mol_type = other DNA
gcgttgctggcgtttttccataggctccgcccccctgacgacgcatcacaaaaatcgacgcgtcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacacgcgtaagacacgacttatcgccactggcagcagccactggtaacaggattagcagagcgaggtatgtaggcggtgctacagagttcttgaagtggtggcctaactac